(19) United States
(12) Patent Application Publication
(10) Pub. No.: US 2015/0031610 A1
(43) Pub. Date: Jan. 29, 2015

(54) DERIVATIZED POLYGLUCOSAMINES FOR DELIVERY OF SMALL MOLECULES, PEPTIDES, AND PROTEINS

(71) Applicant: SYNEDGEN, INC., Claremont, CA (US)

(72) Inventors: Shenda M. Baker, Upland, CA (US); William P. Wiesmann, Washington, DC (US); Ruth Baxter, Los Angeles, CA (US)

(21) Appl. No.: 14/383,449

(22) PCT Filed: Mar. 4, 2013
(86) PCT No.: PCT/US13/28881

§ 371 Date: Sep. 5, 2014

Related U.S. Application Data
(60) Provisional application No. 61/606,922, filed on Mar. 5, 2012.

(51) Int. Cl. A61K 47/36; A61K 31/203; A61K 31/4166; A61K 38/18

(52) U.S. Cl. CPC A61K 47/36; A61K 38/1808; A61K 38/1841; A61K 31/203; A61K 31/4166
USPC 514/8.9; 514/54; 514/9.6; 435/375; 435/184

(57) ABSTRACT

Described herein are compositions comprising a derivatized polyglucosamine and a small molecule, peptide, or protein and related methods of use, e.g., to deliver a small molecule, peptide, or protein to cells (e.g., cancer cells) or tissues (e.g., mucosal membrane and epithelial membrane), e.g., to treat a disease or condition in a subject.

### CROSS REFERENCE TO RELATED APPLICATIONS

This application claims priority of U.S. Application Ser. No. 61/606,922, filed on Mar. 5, 2012, the contents of which are hereby incorporated by reference in their entirety.

### FIELD OF THE INVENTION

The invention relates to derivatized polyglucosamine and its use as a carrier for small molecules, peptides, and proteins, e.g., in the delivery of small molecules, peptides, and proteins to cells (e.g., cancer cells), enzymes, or tissues (e.g., mucosal membrane and epithelial membrane).

### SUMMARY OF THE INVENTION

Compositions comprising derivatized polyglucosamines and a small molecule, peptide, or protein and related methods of use are described herein. Polyglucosamine may be derived from chitosan or chitin. Exemplary methods using the compositions described herein include, but not limited to, methods of delivering small molecules, peptides, or proteins to cells (e.g., cancer cells), enzymes, or tissues (e.g., mucosal membrane and epithelial membrane) in a subject, methods of administering small molecules, peptides, or proteins to a subject, and methods for treating diseases or conditions in a subject. Compositions described herein can also be used to transfect cells with peptides or proteins. Compositions described herein can also be used to activate biological pathways.

In one aspect, a method of delivering a small molecule, peptide, or protein to a cell or enzyme, the method comprising providing a cell or enzyme, providing a composition comprising a small molecule, peptide, or protein and a derivatized polyglucosamine of the following formula (I):

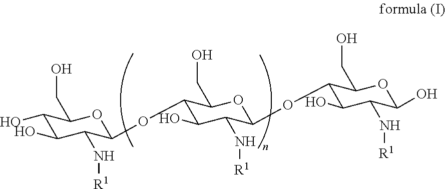

wherein n is an integer between 20 and 6000, and each R¹ is independently selected for each occurrence from hydrogen, acetyl, and either: a) a group of formula (II):

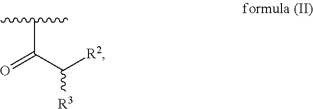

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety, and contacting the composition with the cell or enzyme, thereby delivering the small molecule, peptide, or protein to the cell or enzyme.

In some embodiments, the molecular weight of the derivatized polyglucosamine is between 10,000 and 1,000,000 Da. In some embodiments, the molecular weight of the derivatized polyglucosamine is between 15,000 and 350,000 Da. In some embodiments, the molecular weight of the derivatized polyglucosamine is between 25,000 and 200,000 Da. In some embodiments, the molecular weight of the functionalized polyglucosamine is between 25,000 and 150,000 Da. In some embodiments, the molecular weight of the functionalized polyglucosamine is between 25,000 and 110,000 Da.

In another aspect, a composition comprising a small molecule, peptide, or protein and a derivatized polyglucosamine of the following formula (I):

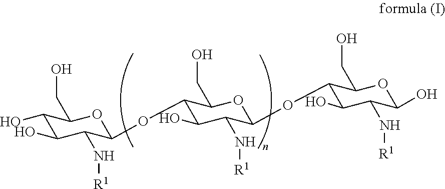

wherein n is an integer between 20 and 6000, and each R¹ is independently selected for each occurrence from hydrogen, acetyl, and either: a) a group of formula (II):

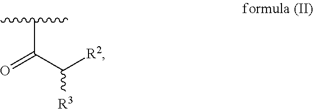

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety (e.g., which can be used to deliver the small molecule, peptide, or protein to a cell, enzyme, or mucosal or epithelial surface).

In another aspect, a small molecule, peptide, or protein/polyglucosamine derivative complex comprising a small molecule, peptide, or protein, and a derivatized polyglucosamine of the following formula (I):

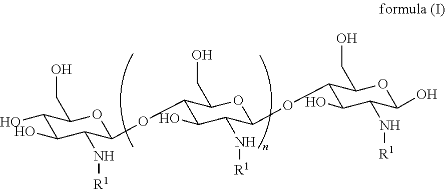

wherein n is an integer between 20 and 6000, and each R¹ is independently selected for each occurrence from hydrogen, acetyl, and either: a) a group of formula (II):

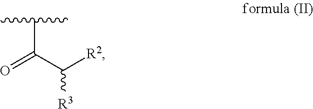

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety (e.g., which can be used to deliver the small molecule, peptide, or protein to a cell, enzyme, or mucosal or epithelial surface).

In another aspect, a method for administering a composition to a subject, the method comprising providing a composition comprising a small molecule, peptide, or protein and a derivatized polyglucosamine of the following formula (I):

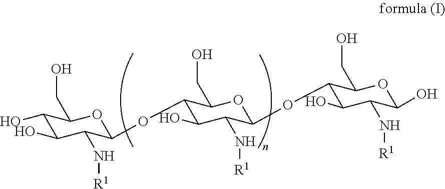

wherein n is an integer between 20 and 6000, and each R¹ is independently selected for each occurrence from hydrogen, acetyl, and either: a) a group of formula (II):

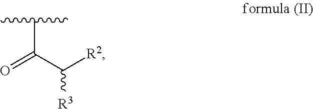

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety and administering an effective amount of the composition to a subject, thereby administering the composition to the subject.

In other aspects, the compositions and complexes described herein may be incorporated into kits or may be used for other methods, e.g., for delivering small molecules, peptides, and proteins to or within cells or for activating biological pathways. In some embodiments, the compositions and complexes described herein allow the administration of a small molecule, peptide, or protein at less than the known effective amount. That is, by administering the smaller than effective amount as part of a composition or complex comprising a derivatized polyglucosamine, the smaller than effective amount results in the same level of response as when an effective amount is administered without derivatized polyglucosamine.

### BRIEF DESCRIPTION OF THE DRAWINGS

FIG. 1 depicts the effect of poly(acetyl, arginyl) glucosamine (PAAG) on ERK activation induced by submaximal concentration of EGF.

FIG. 2 depicts the effect of MEK and EGFR inhibitors on ERK activation induced by EGF in the presence or absence of poly(acetyl, arginyl) glucosamine (PAAG)

FIG. 3 depicts the effect of polyglucosamine derivatives of different molecular weights, degrees of functionalization, and types of modification on EGF-induced ERK phosphorylation.

FIG. 4 depicts the effect of poly(acetyl, arginyl) glucosamine (PAAG) on collagen production in response to TGFβ.

FIG. 5 depicts the effect of arginine on collagen production in response to TGFβ.

FIG. 6 depicts the effect of poly(acetyl, arginyl) glucosamine (PAAG) on collagen production in response to retinoic acid.

FIG. 7 depicts the effect of poly(acetyl, arginyl) glucosamine (PAAG) on collagen production in response to phenytoin.

FIG. 8 depicts the effect of poly(acetyl, arginyl) glucosamine (PAAG) on mammalian cell viability.

### DETAILED DESCRIPTION

Described herein are methods, compositions, complexes, and kits for delivering small molecules, peptides, or proteins, e.g., small molecules, peptides, or proteins described herein, to a cell (e.g., a cancer cell), or enzyme, or tissue (e.g., mucosal membrane and epithelial membrane).

In one aspect, a method of delivering a small molecule, peptide, or protein to a cell or enzyme, the method comprising providing a cell or enzyme, providing a composition comprising a small molecule, peptide, or protein and a derivatized polyglucosamine of the following formula (I):

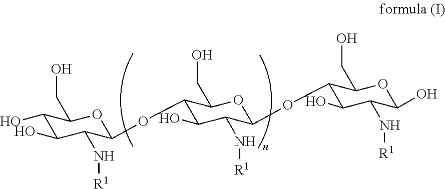

wherein n is an integer between 20 and 6000, and each R¹ is independently selected for each occurrence from hydrogen, acetyl, and either: a) a group of formula (II):

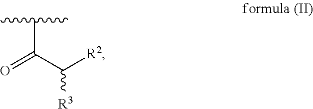

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety, and contacting the composition with the cell or enzyme, thereby delivering the small molecule, peptide, or protein to the cell or enzyme.

In some embodiments, the derivatized polyglucosamine is soluble in aqueous solution from about pH 5.0 to about pH 6.0, e.g., in wounds or duodenum. In some embodiments, the derivatized polyglucosamine is soluble in aqueous solution from about pH 2.0 to about pH 4.0, e.g., in stomach. In some embodiments, the derivatized polyglucosamine is soluble in aqueous solution from about pH 8.0 to about pH 8.5, e.g., in lower part of the gastrointestinal tract.

In another aspect, a method of delivering a small molecule, peptide, or protein to a cell or enzyme, the method comprising providing a cell, contacting the cell or enzyme with a derivatized polyglucosamine of the following formula (I):

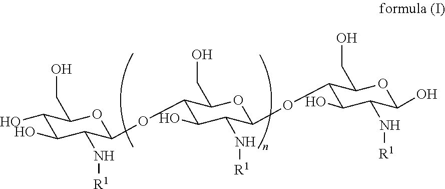

wherein n is an integer between 20 and 6000, and each R¹ is independently selected for each occurrence from hydrogen, acetyl, and either: a) a group of formula (II):

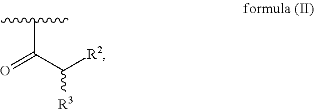

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety, and contacting the cell or enzyme with the small molecule, peptide, or protein, thereby delivering the small molecule, peptide, or protein to the cell or enzyme.

In another aspect, a composition comprising a small molecule, peptide, or protein and a derivatized polyglucosamine of the following formula (I):

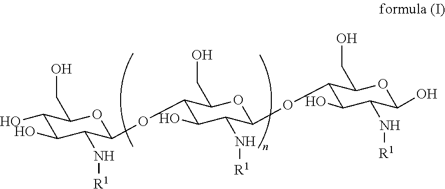

wherein n is an integer between 20 and 6000, and each R¹ is independently selected for each occurrence from hydrogen, acetyl, and either: a) a group of formula (II):

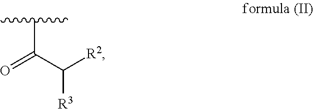

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety (e.g., which can be used to deliver the small molecule, peptide, or protein to a cell, enzyme, or mucosal or epithelial surface).

In another aspect, a small molecule, peptide, or protein/polyglucosamine derivative complex comprising a small molecule, peptide, or protein, and a derivatized polyglucosamine of the following formula (I):

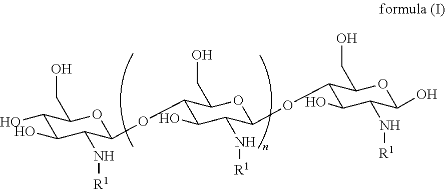

wherein n is an integer between 20 and 6000, and each R¹ is independently selected for each occurrence from hydrogen, acetyl, and either: a) a group of formula (II):

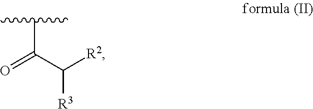

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety (e.g., which can be used to deliver the small molecule, peptide, or protein to a cell, enzyme, or mucosal or epithelial surface).

In another aspect a method of delivering a small molecule, peptide, or protein to or across a mucosal or epithelial surface in a subject, the method comprising providing a composition comprising a small molecule, peptide, or protein and a derivatized polyglucosamine of the following formula (I):

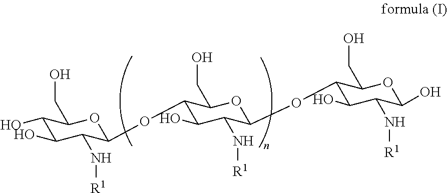

wherein n is an integer between 20 and 6000 and each R¹ is independently selected for each occurrence from hydrogen, acetyl, and either: a) a group of formula (II):

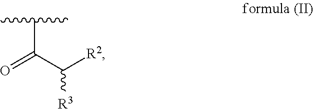

wherein R² is hydrogen or amino, and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety, and contacting the composition with a mucosal surface in a subject, thereby delivering the small molecule, peptide, or protein to or across the mucosal surface in the subject.

In another aspect, a method for administering a composition to a subject, the method comprising providing a composition comprising a small molecule, peptide, or protein and a derivatized polyglucosamine of the following formula (I):

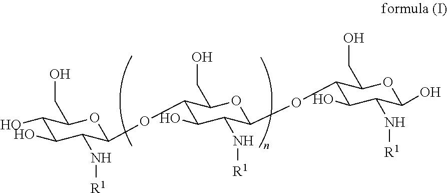

wherein n is an integer between 20 and 6000, and each R¹ is independently selected for each occurrence from hydrogen, acetyl, and either: a) a group of formula (II):

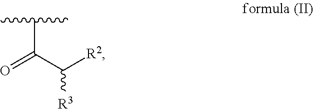

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety and administering an effective amount of the composition to a subject, thereby administering the composition to the subject.

In another aspect, a method for treating a disease or condition, or a symptom of a disease or condition in a subject, the method comprising administering an effective amount of a composition comprising a small molecule, peptide, or protein and a derivatized polyglucosamine of the following formula (I):

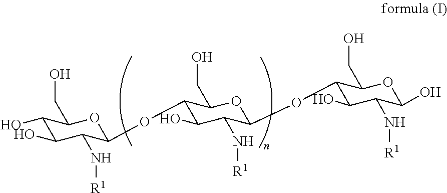

wherein n is an integer between 20 and 6000, and each R¹ is independently selected for each occurrence from hydrogen, acetyl, and either: a) a group of formula (II):

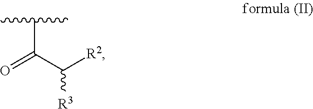

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety to a subject, thereby treating the disease or condition.

In another aspect, a method for treating a subject, the method comprising administering an effective amount of a composition comprising a small molecule, peptide, or protein and a derivatized polyglucosamine of the following formula (I):

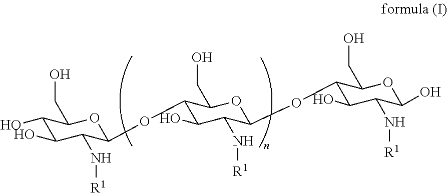

wherein n is an integer between 20 and 6000, and each R¹ is independently selected for each occurrence from hydrogen, acetyl, and either: a) a group of formula (II):

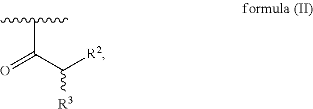

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety to a subject, thereby treating the subject.

In another aspect, a method of delivering a small molecule, peptide, or protein to or within a cell, the method comprising providing a cell, providing a composition comprising a small molecule, peptide, or protein and a derivatized polyglucosamine of the following formula (I):

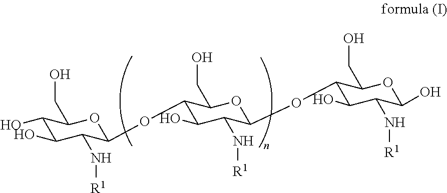

wherein n is an integer between 20 and 6000, and each R¹ is independently selected for each occurrence from hydrogen, acetyl, and either: a) a group of formula (II):

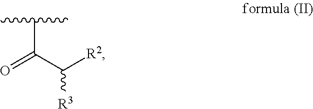

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety and contacting the composition with the cell, thereby delivering the small molecule, peptide, or protein to or within the cell.

In another aspect, a method of delivering a small molecule, peptide, or protein to or within a cell, the method comprising providing a cell, contacting the cell with a derivatized polyglucosamine of the following formula (I):

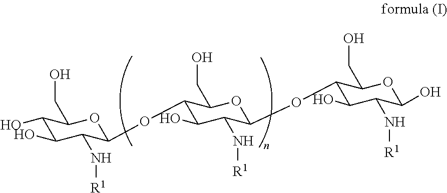

wherein n is an integer between 20 and 6000, and each R¹ is independently selected for each occurrence from hydrogen, acetyl, and either: a) a group of formula (II):

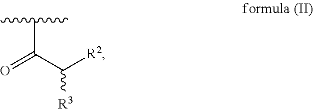

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety and contacting the cell with a small molecule, peptide, or protein, thereby delivering the small molecule, peptide, or protein to or within the cell.

In another aspect, a method of making a composition comprising a small molecule, peptide, or protein and a derivatized polyglucosamine, the method comprising providing a small molecule, peptide, or protein, providing a derivatized polyglucosamine of the following formula (I):

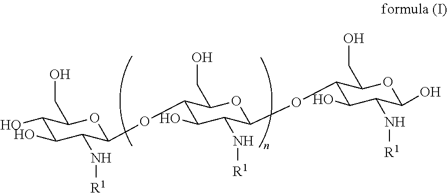

wherein n is an integer between 20 and 6000, and each R¹ is independently selected for each occurrence from hydrogen, acetyl, and either: a) a group of formula (II):

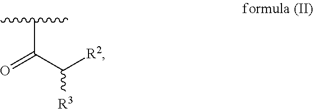

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety and contacting the small molecule, peptide, or protein and the derivatized polyglucosamine,
thereby making the composition.

In another aspect, a kit comprising a small molecule, peptide, or protein and a derivatized polyglucosamine of the following formula (I):

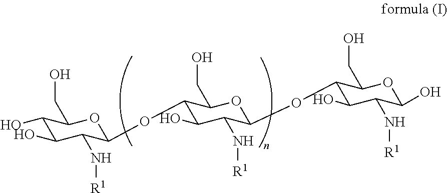

wherein n is an integer between 20 and 6000, and each R¹ is independently selected for each occurrence from hydrogen, acetyl, and either: a) a group of formula (II):

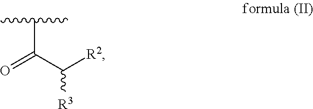

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety (e.g., which can be used to deliver the small molecule, peptide, or protein to a cell, enzyme or mucosal or epithelial surface).

In another aspect a kit comprising a small molecule, peptide, or protein/polyglucosamine derivative complex, wherein the complex comprises a derivatized polyglucosamine of the following formula (I):

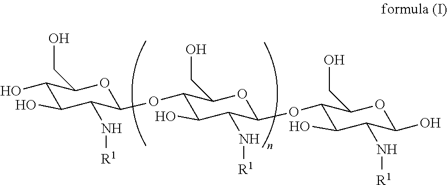

wherein n is an integer between 20 and 6000, and each R¹ is independently selected for each occurrence from hydrogen, acetyl, and either: a) a group of formula (II):

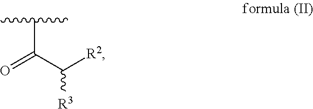

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety (e.g., which can be used to deliver the small molecule, peptide, or protein to a cell, enzyme, or mucosal or epithelial surface).

In one aspect, a method of delivering a small molecule, peptide, or protein to a cell or enzyme, the method comprising providing a cell or enzyme, providing a composition comprising a small molecule, peptide, or protein and a derivatized polyglucosamine of the following formula:

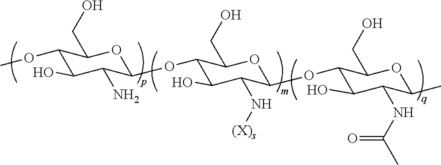

where m is 0.02-0.50; q is 0.50-0.01; s is 1; p+q+m=1; the percentage degree of functionalization is m/(1−q)·100%; and X is selected from the group consisting of:

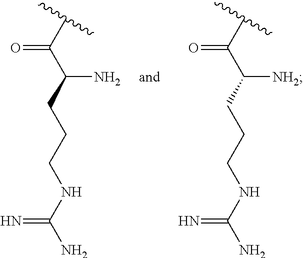 [SMILES: *C(=O)[C@@H](N)CCCNC(=N)N ; *C(=O)[C@H](N)CCCNC(=N)N]

wherein the preparation is substantially free of compounds having a molecular weight of less than 5000 Da, and contacting the composition with the cell or enzyme, thereby delivering the small molecule, peptide, or protein to the cell or enzyme.

In another aspect, a method of delivering a small molecule, peptide, or protein to a cell or enzyme, the method comprising providing a cell, contacting the cell or enzyme with a derivatized polyglucosamine of the following formula:

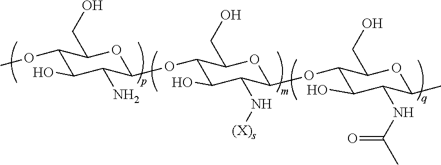

where m is 0.02-0.50; q is 0.50-0.01; s is 1; p+q+m=1; the percentage degree of functionalization is m/(1−q)·100%; and X is selected from the group consisting of:

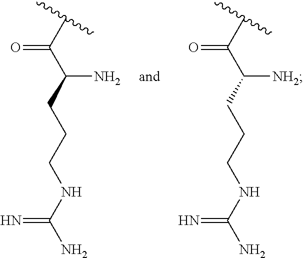 [SMILES: *C(=O)[C@@H](N)CCCNC(=N)N ; *C(=O)[C@H](N)CCCNC(=N)N]

wherein the preparation is substantially free of compounds having a molecular weight of less than 5000 Da, and contacting the cell or enzyme with the small molecule, peptide, or protein, thereby delivering the small molecule, peptide, or protein to the cell or enzyme.

In another aspect, a composition comprising a small molecule, peptide, or protein and a derivatized polyglucosamine of the following formula:

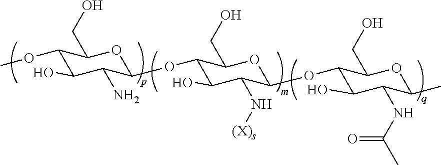

where m is 0.02-0.50; q is 0.50-0.01; s is 1; p+q+m=1; the percentage degree of functionalization is m/(1−q)·100%; and X is selected from the group consisting of:

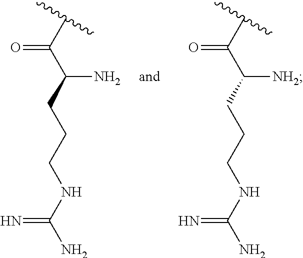 [SMILES: *C(=O)[C@@H](N)CCCNC(=N)N ; *C(=O)[C@H](N)CCCNC(=N)N]

wherein the preparation is substantially free of compounds having a molecular weight of less than 5000 Da (e.g., which can be used to deliver the small molecule, peptide, or protein to a cell, enzyme, or mucosal or epithelial surface).

In another aspect, a small molecule, peptide, or protein/polyglucosamine derivative complex comprising a small molecule, peptide, or protein, and a derivatized polyglucosamine of the following formula:

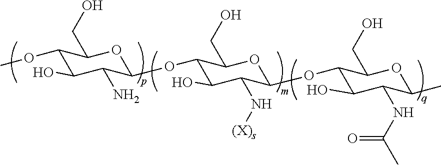

where m is 0.02-0.50; q is 0.50-0.01; s is 1; p+q+m=1; the percentage degree of functionalization is m/(1−q)·100%; and X is selected from the group consisting of:

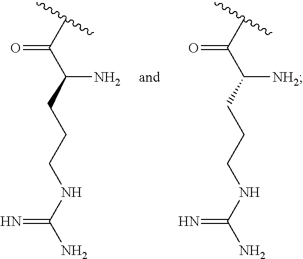 [SMILES: *C(=O)[C@@H](N)CCCNC(=N)N ; *C(=O)[C@H](N)CCCNC(=N)N]

wherein the preparation is substantially free of compounds having a molecular weight of less than 5000 Da (e.g., which can be used to deliver the small molecule, peptide, or protein to a cell, enzyme, or mucosal or epithelial surface).

In another aspect, a method for treating a disease or condition, or a symptom of a disease or condition in a subject, the method comprising administering an effective amount of a composition comprising a small molecule, peptide, or protein and a derivatized polyglucosamine of the following formula:

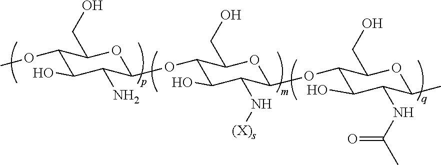

where m is 0.02-0.50; q is 0.50-0.01; s is 1; p+q+m=1; the percentage degree of functionalization is m/(1−q)·100%; and X is selected from the group consisting of:

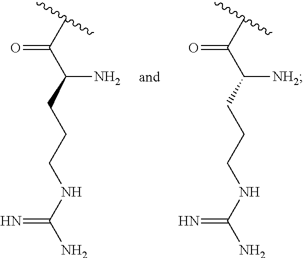 [SMILES: *C(=O)[C@@H](N)CCCNC(=N)N ; *C(=O)[C@H](N)CCCNC(=N)N]

wherein the preparation is substantially free of compounds having a molecular weight of less than 5000 Da to a subject, thereby treating the disease or condition.

In another aspect, a method of delivering a small molecule, peptide, or protein to or within a cell, the method comprising providing a cell, contacting the cell with a derivatized polyglucosamine of the following formula:

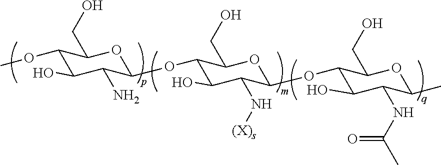

where m is 0.02-0.50; q is 0.50-0.01; s is 1; p+q+m=1; the percentage degree of functionalization is m/(1−q)·100%; and X is selected from the group consisting of:

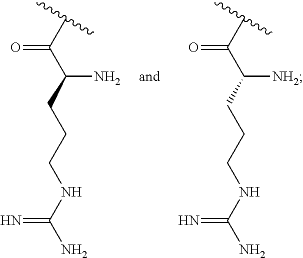 [SMILES: *C(=O)[C@@H](N)CCCNC(=N)N ; *C(=O)[C@H](N)CCCNC(=N)N]

wherein the preparation is substantially free of compounds having a molecular weight of less than 5000 Da and contacting the cell with a small molecule, peptide, or protein, thereby delivering the small molecule, peptide, or protein to or within the cell.

In another aspect, a method of making a composition comprising a small molecule, peptide, or protein and a derivatized polyglucosamine, the method comprising providing a small molecule, peptide, or protein, providing a derivatized polyglucosamine of the following formula:

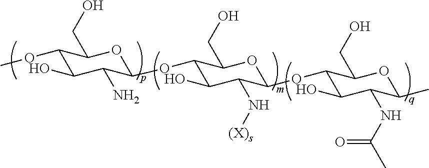

where m is 0.02-0.50; q is 0.50-0.01; s is 1; p+q+m=1; the percentage degree of functionalization is m/(1−q)·100%; and X is selected from the group consisting of:

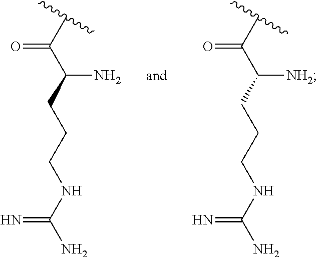 [SMILES: *C(=O)[C@@H](N)CCCNC(=N)N ; "and" ; *C(=O)[C@H](N)CCCNC(=N)N]

wherein the preparation is substantially free of compounds having a molecular weight of less than 5000 Da and contacting the small molecule, peptide, or protein and the derivatized polyglucosamine, thereby making the composition.

In another aspect, a kit comprising a small molecule, peptide, or protein and a derivatized polyglucosamine of the following formula:

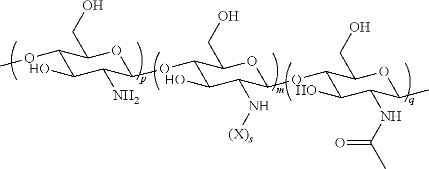

where m is 0.02-0.50; q is 0.50-0.01; s is 1; p+q+m=1; the percentage degree of functionalization is m/(1−q)·100%; and X is selected from the group consisting of:

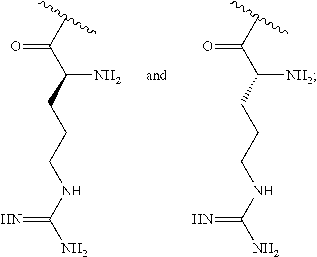 [SMILES: *C(=O)[C@@H](N)CCCNC(=N)N ; "and" ; *C(=O)[C@H](N)CCCNC(=N)N]

wherein the preparation is substantially free of compounds having a molecular weight of less than 5000 Da (e.g., which can be used to deliver the small molecule, peptide, or protein to a cell, enzyme or mucosal or epithelial surface).

In another aspect, a method of activating a biological pathway, the method comprising providing a cell or an enzyme, providing a composition comprising a small molecule, peptide, or protein and a derivatized polyglucosamine of the following formula (I):

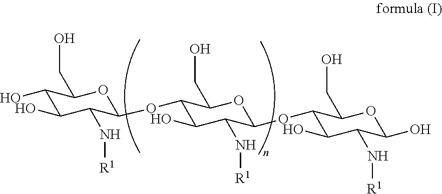

wherein n is an integer between 20 and 6000, and each R¹ is independently selected for each occurrence from hydrogen, acetyl, and either: a) a group of formula (II):

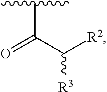

wherein R² is hydrogen or amino and R³ is amino, guanidino, C₁-C₆ alkyl substituted with an amino or guanidino moiety, or a natural or unnatural amino acid side chain; or b) R¹, when taken together with the nitrogen to which it is attached, forms a guanidine moiety, wherein at least 25% of R¹ substituents are H, at least 1% of R¹ substituents are acetyl, and at least 2% of R¹ substituents are a group of formula (II) or are taken together with the nitrogen to which they are attached to form a guanidine moiety and contacting the composition with the cell or enzyme, thereby activating the biological pathway.

In another aspect, a method of activating a biological pathway, the method comprising providing a cell or an enzyme, providing a composition comprising a small molecule, peptide, or protein and a derivatized polyglucosamine of the following formula:

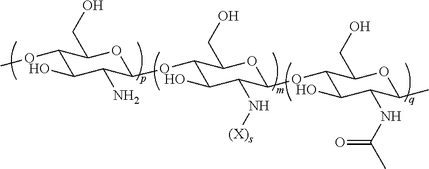

where m is 0.02-0.50; q is 0.50-0.01; s is 1; p+q+m=1; the percentage degree of functionalization is m/(1−q)·100%; and X is selected from the group consisting of:

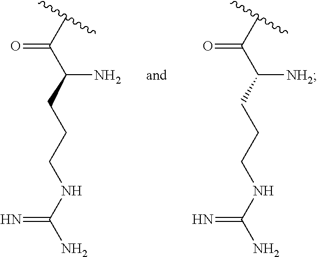 [SMILES: *C(=O)[C@@H](N)CCCNC(=N)N ; "and" ; *C(=O)[C@H](N)CCCNC(=N)N]

wherein the preparation is substantially free of compounds having a molecular weight of less than 5000 Da and contacting the composition with the cell or enzyme, thereby activating the biological pathway.

In the aspects described above, the compositions and complexes described may allow the administration of a small molecule, peptide, or protein at less than the known effective amount. That is, by administering the smaller than effective amount as part of a composition or complex comprising a derivatized polyglucosamine, the smaller than effective amount results in the same level of response as when an effective amount is administered without derivatized polyglucosamine.

### DEFINITIONS

As used herein, the term “small molecule” refers to a small organic compound having a molecular weight less than, or equal to, about 1000 Da. Some small molecules have therapeutic functionality. They may alleviate, moderate, inhibit, or diminish a disease or disorder. The efficacy of a small molecule may result from inhibition, activation, or modification of a biological pathway related to the disease or disorder. For example, a small molecule may cause the death of rapidly-dividing cells in an organism or in a tissue sample or cell culture. Small molecules include, e.g., anti-cancer compounds, anti-inflammatory compounds, anti-epileptics, pain-relief compounds (analgesics), hormones, anesthetics, antibiotics, antivirals, antifungals, metabolites, and cardiovascular compounds.

As used herein, the term “peptide” refers to a compound made up of a single chain of D- or L-amino acids or a mixture of D- and L-amino acids joined by peptide bonds. Generally, peptides contain at least two amino acid residues and are less than about 50 amino acid residues in length.

As used herein, the term “protein” refers to a compound that is composed of linearly arranged amino acids linked by peptide bonds, but in contrast to peptides, has a well-defined conformation. Generally, proteins consist of one or more chains of 50 or more amino acid residues. Proteins may include, e.g., growth factors (EGF, TGF-α, TGF-β, TNF, HGF, IGF, IL-1-8, etc.) cytokines, paratopes, Fabs (fragments, antigen binding), and antibodies.

As used herein, the term “therapeutic peptide” refers to a peptide comprising two or more amino acids, covalently linked together through one or more amide bonds, wherein upon administration of the peptide to a subject, the subject receives a therapeutic effect (e.g., administration of the therapeutic peptide treats a cell, or cures, alleviates, relieves or improves a symptom of a disorder). A therapeutic peptide may comprise, e.g., more than two, three, four, five, six, seven, eight, nine, ten, eleven, twelve, thirteen, fourteen, fifteen amino acids. In some embodiments, a therapeutic peptide comprises more than 15, e.g., greater than 20, 25, 30, 35, 40, 45, 50, 55, 60, 65, 70, 75, 80, 85, or 90 amino acids. For example, in some embodiments, the therapeutic peptide is more than 5, 7, 9, 10, 11 or 12 amino acids in length.

### Polyglucosamine Derivatives

The compositions described herein include a functionalized polyglucosamine derivative and a small molecule, peptide, or protein.

Polyglucosamines can be derived from chitosan by deacetylation. Chitosan is an insoluble polymer derived from chitin, which is a polymer of N-acetylglucosamine that is the main component of the exoskeletons of crustaceans (e.g. shrimp, crab, lobster). Polyglucosamines are also found in various fungi and arthropods. Synthetic sources and alternate sources of β1-4 polyglucosamines ma serve as the starting material for the polyglucosamine derivatives. The polyglucosamine derivatives described herein are generated by functionalizing the free amino groups with positively charged or neutral moieties, as described herein. Up to 50% of the amino groups are acetylated. For the purposes of this invention, if greater than 50% of the amino groups are acetylated, the polymer is considered a polyacetylglucosamine. The degrees of deacetylation and functionalization impart a specific charge density to the functionalized polyglucosamine derivative. The resulting charge density affects solubility, and the strength of interaction with cell membranes. The molecular weight is also an important factor in the tenacity of cell membrane interaction and thus drug delivery capacity. Thus, in accordance with the present invention, the degree of deacetylation, the functionalization and the molecular weight must be optimized for optimal efficacy. The derivatized polyglucosamines described herein have a number of properties which are advantageous including solubility at physiologic pH and drug delivery capacity when in solution at pH less than about 9.

A soluble polyglucosamine as described herein, refers to a water soluble chitosan or polyglucosamine that is not derivatized on the hydroxyl or amine moieties other than with acetyl groups. A soluble polyglucosamine is comprised of glucosamine and acetylglucosamine monomers. Generally a water soluble polyglucosamine has a molecular weight of less than or equal to about 1000 kDa and a degree of deacetylation equal or greater than 80%. In some embodiments, the molecular weight of the soluble polyglucosamine is between 10,000 and 1,000,000 Da. In some embodiments, the molecular weight of the soluble polyglucosamine is between 15,000 and 350,000 Da. In some embodiments, the molecular weight of the soluble polyglucosamine is between 25,000 and 200,000 Da. In some embodiments, the molecular weight of the soluble polyglucosamine is between 25,000 and 150,000 Da. In some embodiments, the molecular weight of the soluble polyglucosamine is between 25,000 and 110,000 Da. The soluble polyglucosamines described herein are soluble at neutral and physiological pH. Water soluble is defined as being fully dissolvable in water at pH 7.

The polyglucosamine derivatives described herein are generated by functionalizing the resulting free amino groups with positively charged Or neutral moieties, as described herein.

Polyglucosamines with any degree of deacetylation (DDA) greater than 50% are used in the present invention, with functionalization between 2% and 50% of the available amines. The degree of deacetylation determines the relative content of free amino groups to total monomers in the polyglucosamine polymer. Methods that can be used for determination of the degree of deacetylation of polyglucosamine include, e.g., ninhydrin test, linear potentiometric titration, near-infrared spectroscopy, nuclear magnetic resonance spectroscopy, hydrogen bromide titrimetry, infrared spectroscopy, and first derivative UV-spectrophotometry. Preferably, the degree of deacetylation of a soluble polyglucosamine or a derivatized polyglucosamine described herein is determined by quantitative infrared spectroscopy. Percent functionalization is determined as the % of derivatized amines relative to the total number of available amino moieties prior to reaction on the polyglucosamine polymer. Preferably, the percent functionalization of a derivatized polyglucosamine described herein is determined by H-NMR or quantitative elemental analysis. The degrees of deacetylation and functionalization impart a specific charge density to the functionalized polyglucosamine derivative. The resulting charge density affects solubility, and strength of interaction with cell membranes. The molecular weight is also an important factor in the tenacity of cell membrane interaction and thus drug delivery capacity. Thus, in accordance with the present invention, these properties must be optimized for optimal efficacy. Exemplary polyglucosamine derivatives are described in U.S. Pat. No. 8,119,780, which is incorporated herein by reference in its entirety.

The polyglucosamine derivatives described herein have a range of polydispersity index (PDI) between about 1.0 to about 2.5. As used herein, the polydispersity index (PDI), is a measure of the distribution of molecular weights in a given polymer sample. The PDI calculated is the weight averaged molecular weight divided by the number averaged molecular weight. This calculation indicates the distribution of individual molecular weights in a batch of polymers. The PDI has a value always greater than 1, but as the polymer chains approach uniform chain length, the PDI approaches unity (1). The PDI of a polymer derived from a natural source depends on the natural source (e.g. chitin or chitosan from crab vs. shrimp vs. fungi) and can be affected by a variety of reaction, production, processing, handling, storage and purifying conditions. Methods to determine the polydispersity include, e.g., gel permeation chromatography (also known as size exclusion chromatography); light scattering measurements; and direct calculation from MALDI or from electrospray mass spectrometry. Preferably, the PDI of a soluble polyglucosamine or a derivatized polyglucosamine described herein is determined by HPLC and multi angle light scattering methods.

The polyglucosamine derivatives (i.e., derivatized polyglucosamines) described herein have a variety of selected molecular weights that are soluble at neutral and physiological pH, and include for the purposes of this invention molecular weights ranging from 5-1,000 kDa. Embodiments described herein are feature medium range molecular weight of derivatized polyglucosamines (25 kDa, e.g., from about 15 to about 300 kDa) which can have drug delivery properties. In some embodiments, the molecular weight of the derivatized polyglucosamine is between 10,000 and 1,000,000 Da. In some embodiments, the molecular weight of the derivatized polyglucosamine is between 15,000 and 350,000 Da. In some embodiments, the molecular weight of the derivatized polyglucosamine is between 25,000 and 200,000 Da. In some embodiments, the molecular weight of the functionalized polyglucosamine is between 25,000 and 150,000 Da. In some embodiments, the molecular weight of the functionalized polyglucosamine is between 25,000 and 110,000 Da.

The functionalized polyglucosamine derivatives described herein include the following:

(A) Polyglucosamine-arginine compounds;

(B) Polyglucosamine-natural amino acid derivative compounds;

(C) Polyglucosamine-unnatural amino acid compounds;

(D) Polyglucosamine-acid amine compounds;

(E) Polyglucosamine-guanidine compounds; and

(F) Neutral polyglucosamine derivative compounds.

(A) Polyglucosamine-Arginine Compounds

In some embodiments, the present invention is directed to polyglucosamine-arginine compounds, where the arginine is bound through a peptide (amide) bond via its carbonyl to the primary amine on the glucosamines of polyglucosamine:

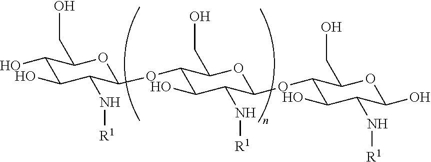

wherein each R¹ is independently selected from hydrogen, acetyl, and a group of the following formula:

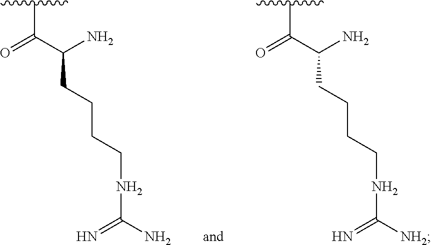 [SMILES: *C(=O)[C@@H](N)CCCCNC(=N)N ; "and" ; *C(=O)[C@H](N)CCCCNC(=N)N]

or a racemic mixture thereof,

wherein at least 25% of R¹ substituents are H, at least 1% are acetyl, and at least 2% are a group of the formula shown above.

In some embodiments, a polyglucosamine-arginine compound is of the following formula

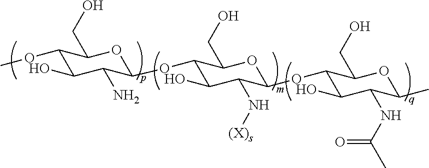

where m is 0.02-0.50; q is 0.50-0.01; s is 1; p+q+m=1; the percentage degree of functionalization is m/(1−q)·100%; and X is selected from the group consisting of:

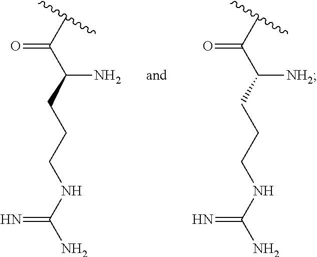 [SMILES: *C(=O)[C@@H](N)CCCNC(=N)N ; "and" ; *C(=O)[C@H](N)CCCNC(=N)N]

wherein the preparation is substantially free of compounds having a molecular weight of less than 5000 Da

(B) Polyglucosamine-Natural Amino Acid Derivative Compounds

In some embodiments, the present invention is directed to polyglucosamine-natural amino acid derivative compounds, wherein the natural amino acid may be histidine or lysine. The amino is bound through a peptide (amide) bond via its carbonyl to the primary amine on the glucosamines of polyglucosamine:

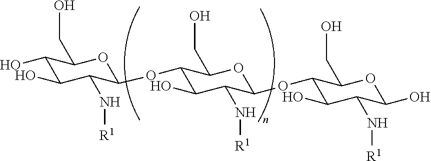

wherein each R¹ is independently selected from hydrogen, acetyl, and a group of the following formula:

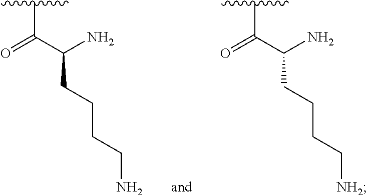 [SMILES: *C(=O)[C@@H](N)CCCCN ; "and" ; *C(=O)[C@H](N)CCCCN]

or a racemic mixture thereof, wherein at least 25% of R¹ substituents are H, at least 1% are acetyl, and at least 2% are a group of the formula shown above; or a group of the following formula:

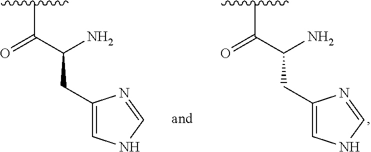 [SMILES: *C(=O)[C@@H](N)Cc1c[nH]cn1 ; *C(=O)[C@H](N)Cc1c[nH]cn1]

or a racemic mixture thereof, wherein at least 25% of R¹ substituents are H, at least 1% are acetyl, and at least 2% are a group of the formula shown above.

(C) Polyglucosamine-Unnatural Amino Acid Compounds

In some embodiments, the present invention is directed to polyglucosamine-unnatural amino acid compounds, where the unnatural amino acid is bound through a peptide (amide) bond via its carbonyl to the primary amine on the glucosamines of polyglucosamine:

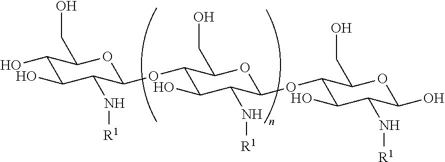

wherein each R¹ is independently selected from hydrogen, acetyl, and a group of the following formula:

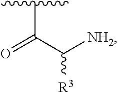

wherein R³ is an unnatural amino acid side chain, and wherein at least 25% of R¹ substituents are H, at least 1% are acetyl, and at least 2% are a group of the formula shown above.

Unnatural amino acids are those with side chains not normally found in biological systems, such as ornithine (2,5-diaminopentanoic acid). Any unnatural amino acid may be used in accordance with the invention. In some embodiments, the unnatural amino acids coupled to polyglucosamine have the following formulae:

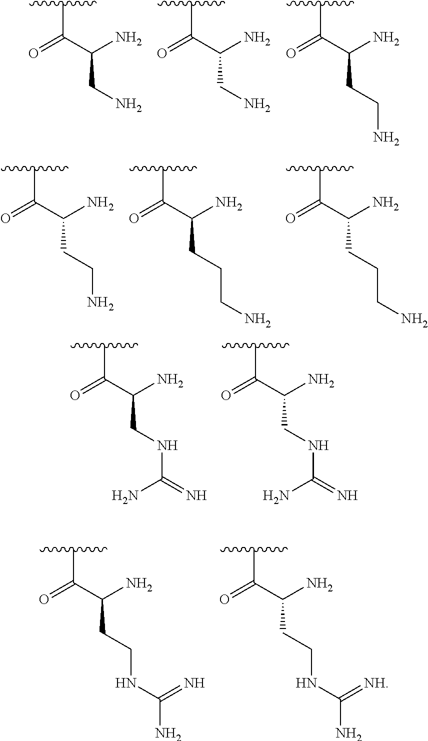

(D) Polyglucosamine-Acid Amine Compounds

In some embodiments, the present invention is directed to polyglucosamine-acid amine compounds, or their guanidylated counterparts. The acid amine is bound through a peptide (amide) bond via its carbonyl to the primary amine on the glucosamines of polyglucosamine:

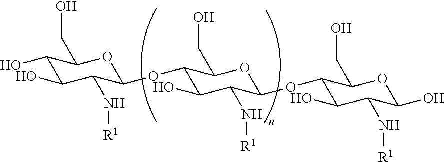

wherein each R¹ is independently selected from hydrogen, acetyl, and a group of the following formula:

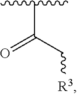

wherein R³ is selected from amino, guanidino, and C₁-C₆ alkyl substituted with an amino or a guanidino group, wherein at least 25% of R¹ substituents are H, at least 1% are acetyl, and at least 2% are a group of the formula shown above

In some embodiments, R¹ is selected from one of the following:

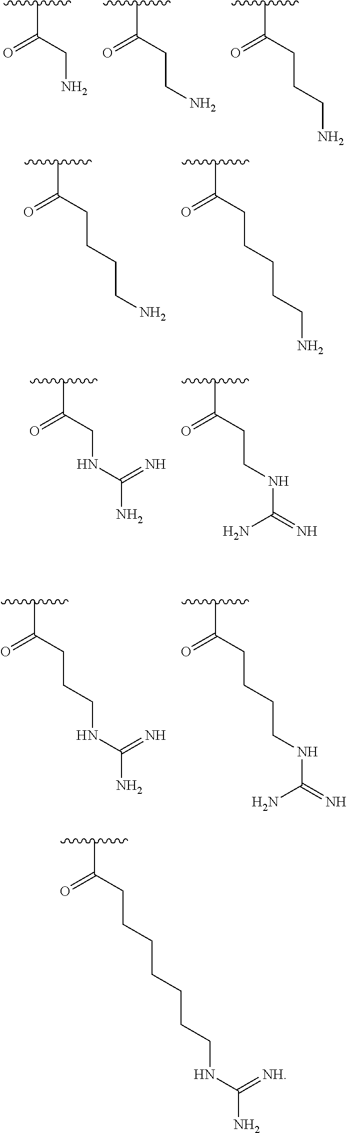

(E) Polyglucosamine-Guanidine Compounds

In some embodiments, the present invention is directed to polyglucosamine-guanidine compounds.

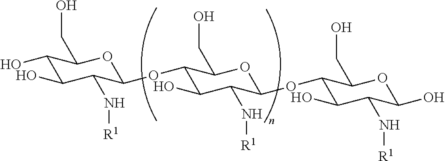

wherein each R¹ is independently selected from hydrogen, acetyl, and a group in which R¹, together with the nitrogen to which it is attached, forms a guanidine moiety; wherein at least 25% of R¹ substituents are H, at least 1% are acetyl, and at least 2% form a guanidine moiety together with the nitrogen to which it is attached.

(F) Neutral Polyglucosamine Derivative Compounds

In some embodiments, the present invention is directed to neutral polyglucosamine derivative compounds. Exemplary neutral polyglucosamine derivative compounds include those where one or more amine nitrogens of the polyglucosamine have been covalently attached to a neutral moiety such as a sugar:

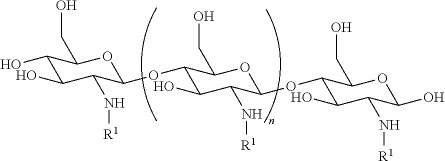

wherein each R¹ is independently selected from hydrogen, acetyl, and a sugar (e.g., a naturally occurring or modified sugar) or an α-hydroxy acid. Sugars can be monosaccharides, disaccharides or polysaccharides such as glucose, mannose, lactose, maltose, cellubiose, sucrose, amylose, glycogen, cellulose, gluconate, or pyruvate. Sugars can be covalently attached via a spacer or via the carboxylic acid, ketone or aldehyde group of the terminal sugar. Examples of α-hydroxy acids include glycolic acid, lactic acid, and citric acid. In some preferred embodiments, the neutral polyglucosamine derivative is polyglucosamine-lactobionic acid compound or polyglucosamine-glycolic acid compound. Exemplary salts and coderivatives include those known in the art, for example, those described in US 2007/0281904, the contents of which is incorporated by reference in its entirety.

### Small Molecules

Methods, compounds and compositions for binding and delivering a small molecule, e.g., to a cell (e.g., a cancer cell), an enzyme, or tissue (e.g., mucosal membrane and epithelial membrane) are described herein. Also described herein are methods, compounds and compositions for administering a small molecule to a subject, e.g., to treat a disorder or condition, or a symptom of a disorder or condition, e.g., pain, inflammatory disorder, proliferative disorder (e.g., cancer), dermal disorder or condition (e.g., wound).

The small molecules described herein include, for example, pharmaceutical compounds, natural products, steroids, opiates, and variants and derivatives thereof, which can be used in the compositions, complexes and particles described herein to treat a disorder or condition, or a symptom thereof, e.g., pain, inflammatory disorder, proliferative disorder (e.g., cancer), dermal disorder or condition (e.g., wound). Small molecules include, e.g., antibiotics, anti-virals, anesthetics, steroidal agents, anti-cancer agents, anti-inflammatory agents (e.g., a non-steroidal anti-inflammatory agents), anti-neoplastic agents, antigens, vaccines, decongestants, antihypertensives, sedatives, birth control agents, progestational agents, anti-cholinergics, analgesics, anti-depressants, anti-psychotics, p-adrenergic blocking agents, diuretics, cardiovascular active agents, vasoactive agents, nutritional agents, and vitamins (e.g., riboflavin, nicotinic acid, pyridoxine, pantothenic acid, biotin, choline, inositol, carnitine, vitamin C, vitamin A, vitamin E, vitamin K). In some embodiments, the small molecule is a vitamin metabolite, e.g., retinoic acid. In some embodiments, the small molecule is an anti-epileptic, e.g., phenytoin (Dilantin™). In some embodiments, the small molecule is polarizable or has a charge, e.g., a negative charge. In some embodiments, the small molecule comprises a moiety that is charged, e.g., negatively charged.

### Therapeutic Peptides

Methods, compounds and compositions for binding and delivering a therapeutic peptide, e.g., to a cell (e.g., a cancer cell), an enzyme, or tissue (e.g., mucosal membrane and epithelial membrane) are described herein. Also described herein are methods, compounds and compositions for administering a therapeutic peptide to a subject, e.g., to treat a disorder or condition, or a symptom of a disorder or condition, e.g., pain, inflammatory disorder, proliferative disorder (e.g., cancer), dermal disorder or condition (e.g., wound).

The therapeutic peptides described herein include, for example, a peptide, and variants and derivatives thereof, which can be used in the compositions, complexes and particles described herein to treat a disorder or condition, or a symptom thereof, e.g., pain, inflammatory disorder, proliferative disorder (e.g., cancer), dermal disorder or condition (e.g., wound).

In some embodiments, the peptide is a linear peptide. In some embodiments, the peptide is a cyclic peptide. In some embodiments, the peptide is a polymeric peptide.

In some embodiments, the peptide comprises less than or equal to about 50, 45, 40, 35, 30, 25, 20, 15, 10, 5, or 2 amino acid residues. In some embodiments, the therapeutic peptide is polarizable or has a charge, e.g., a negative charge. In some embodiments, the therapeutic peptide comprises a moiety that is charged, e.g., negatively charged. In some embodiments that therapeutic peptide comprises one or more amino acid residues which result in a charged therapeutic peptide.

### Therapeutic Proteins

Methods, compounds and compositions for binding and delivering a therapeutic protein, e.g., to a cell (e.g., a cancer cell), an enzyme, or tissue (e.g., mucosal membrane and epithelial membrane) are described herein. Also described herein are methods, compounds and compositions for administering a therapeutic protein to a subject, e.g., to treat a disorder or condition, and/or a symptom of a disorder or a condition, e.g., pain, inflammatory disorder, proliferative disorder (e.g., cancer), dermal disorder or condition (e.g., wound).

The therapeutic peptides described herein include, for example, a protein, e.g., a therapeutic protein, and variants and derivatives thereof, which can be used in the compositions, complexes and particles described herein to treat a disorder or condition, or a symptom thereof, e.g., pain, inflammatory disorder, proliferative disorder (e.g., cancer), dermal disorder or condition (e.g., wound). In some embodiments, the therapeutic protein is polarizable or has a charge, e.g., a negative charge. In some embodiments, the therapeutic protein comprises a moiety that is charged, e.g., negatively charged. In some embodiments that therapeutic protein comprises one or more amino acid residues which result in a charged therapeutic protein.

Exemplary therapeutic proteins include, but not limited to, an analgesic protein, an anti-inflammatory protein, an anti-proliferative protein, an proapoptotic protein, an anti-angiogenic protein, a cytotoxic protein, a cytostatic protein, a cytokine, a chemokine, a growth factor, a wound healing protein, a pharmaceutical protein, or a pro-drug activating protein. Therapeutic proteins may include growth factors (EGF, TGF-α, TGF-β, TNF, HGF, IGF, IL-1-8, etc.) cytokines, paratopes, Fabs (fragments, antigen binding), and antibodies.

In some embodiments, the therapeutic peptide is a growth factor which modulates a biological pathway. In some embodiments, the therapeutic peptide is selected from the group comprising EGF, TGF-α, TGF-β, TNF, HGF, IGF, IL-1, IL-2, IL-3, IL-4, IL-5, IL-6, IL-7, IL-8,

As used herein, the term “analgesic protein” refers to a protein, the presence of which in the target cell or tissue is capable of suppressing pain. Exemplary analgesic proteins include, but not limited to, prostatic acid phosphatase (PAP).

As used herein, the term “anti-inflammatory protein” refers to a protein, the presence of which in the target cell or tissue is capable of reducing acute or chronic inflammatory response. Exemplary anti-inflammatory proteins include, but not limited to, transforming growth factor-β (TGF-β).

As used herein, the term “anti-proliferative protein” refers to a protein, the presence of which in the target cell or tissue is capable of suppressing a proliferative phenotype (e.g., a neoplastic phenotype) and/or inducing apoptosis.

As used herein, the term “proapoptotic protein” refers to a protein, the presence of which in the target cell or tissue is capable of inducing apoptosis or the programmed cell death pathway of the cell. Exemplary apoptotic proteins include, but not limited to, adenovirus E3 and E4 proteins, p53 pathway proteins, and caspases.

As used herein, the term “anti-angiogenic protein” refers to a protein, the presence of which in the target cell or tissue is capable of suppressing angiogenesis or resulting in the extracellular secretion of anti-angiogenic factors. Exemplary anti-angiogenesis factors include angiostatin, inhibitors of vascular endothelial growth factor (VEGF) such as Tie 2, and endostatin.

As used herein, the term “cytotoxic protein” refers to a protein, the presence of which in the target cell or tissue produces a toxic effect. Exemplary cytotoxic proteins include, but not limited to, pseudomonas exotoxin, ricin toxin, and diphtheria toxin.

As used herein, the term “cytostatic protein” refers to a protein, the presence of which in the target cell or tissue produces an arrest in the cell cycle. Exemplary cytostatic genes include, but not limited to, p21, Rb, E2F, cyclin-dependent kinase inhibitors (e.g., p16, p15, p18 and p19), and the growth arrest specific homeobox (GAX) protein.

As used herein, the term “cytokine” refers to a protein that is secreted by specific cells of the immune system and glial cells, which carries signals locally between cells, and thus has an effect on other cells

As used herein, the term “chemokine” refers to a group of structurally related low-molecular weight factors secreted by cells having mitogenic, chemotactic or inflammatory activities. These proteins can be sorted into two groups based on the spacing of the two amino-terminal cysteines. In the first group, the two cysteines are separated by a single residue (C-x-C), while in the second group, they are adjacent (C-C). Examples of member of the ‘C-x-C’ chemokines include, e.g., platelet factor 4 (PF4), platelet basic protein (PBP), interleukin-8 (IL-8), melanoma growth stimulatory activity protein (MGSA), macrophage inflammatory protein 2 (MIP-2), mouse Mig (m119), chicken 9E3 (or pCEF-4), pig alveolar macrophage chemotactic factors I and II (AMCF-I and -II), pre-B cell growth stimulating factor (PBSF), and IP10. Examples of members of the ‘C-C’ group include, e.g., monocyte chemotactic protein 1 (MCP-1), monocyte chemotactic protein 2 (MCP-2), monocytechemotactic protein 3 (MCP-3), monocyte chemotactic protein 4 (MCP-4), macrophage inflammatory protein 1α (MIP-1-α), macrophage inflammatory protein 1β. (MIP-1-β), macrophage inflammatory protein 1-γ (MIP-1-γ), macrophage inflammatory protein 3α (MIP-3-α, macrophage inflammatory protein 3β (MIP-3-β), chemokine (ELC), macrophage inflammatory protein-4 (MIP-4), macrophage inflammatory protein 5 (MIP-5), LD78β, RANTES, SIS-epsilon (p500), thymus and activation-regulated chemokine (TARC), eotaxin, I-309, human protein HCC-1/NCC-2, human protein HCC-3, mouse protein C10.

As used herein, the term “growth factor” refers to a protein that is capable of stimulating cellular growth. Exemplary growth factors include, but not limited to, epidermal growth factor (EGF), platelet-derived growth factor (PDGF), transforming growth factor-α (TGF-α), transforming growth factor-α (TGF-β), fibroblast growth factor (FGF), nerve growth factor (NGF), erythropoietin, insulin-like growth factor-1 (IGF-1), and insulin-like growth factor-2 (IGF-2).

As used herein, the term “wound healing protein” refers to a protein having an effect of wound healing in the target cell or tissue. Exemplary wound healing proteins include, but not limited to, transforming growth factor-α (TGF-α) and transforming growth factor-α (TGF-β).

As used herein, the term “pharmaceutical protein” refers to a protein having pharmaceutically effect in the target cell or tissue. Exemplary pharmaceutical proteins include, but not limited to, insulin, growth hormone, dopamine, serotonin, epidermal growth factor, GABA, ACTH, NGF, VEGF, and thrombospondin. Also, the pharmaceutical proteins may encompass immunoreactive proteins such as antibodies, Fab fragments, Fv fragments, humanized antibodies, chimeric antibodies, single chain antibodies, and human antibodies derived from non-human sources.

As used herein, the term “pro-drug activating protein” refers to a protein, the presence of which in the target cell or tissue, is capable of converting a non-therapeutic compound into a therapeutic compound, which renders the cell susceptible to killing by external factors or causes a toxic condition in the cell. Exemplary pro-drug activating proteins include, but not limited to, cytosine deaminase and thymidine kinase (TK).

### Methods of Making Derivatized Polyglucosamine/Therapeutic Agent Complexes

The polyglucosamine derivative/therapeutic agent (e.g., small molecule, peptide, or protein) complexes described herein can be made (e.g., formed) by various methods. The preparation of polyglucosamine derivatives, more specifically polyglucosamine-arginine derivatives, can be found in a number of earlier publications. See, e.g., U.S. Patent Application Publication Numbers 2007/0281904, 2010/0056474, 2010/0130443, 2010/0137193 and PCT Published Application Numbers WO2010/088565A1, WO2011/028967A1, WO2011/028968A1, WO2011/127144A1, all of which are incorporated by reference in their entireties.

In some embodiments, the polyglucosamine derivative/therapeutic agent complex comprises a particle, wherein the particle comprises a polyglucosamine derivative, and a therapeutic agent (e.g., a small molecule, peptide, or protein). In some embodiments, the particle is nanometers in dimension, for example, due to the nature of the molecules involved, e.g. the polyglucosamine derivative and/or the therapeutic agent (e.g., a small molecule, peptide, or protein described herein).

In one embodiment, the polyglucosamine derivative/therapeutic agent complex is made (e.g., formed) by mixing a polyglucosamine derivative (e.g., a polyglucosamine derivative described herein (e.g., polyglucosamine-arginine)) with a therapeutic agent (e.g., a small molecule, peptide, or protein described herein), e.g., at a weight ratio (polyglucosamine derivative:therapeutic agent) of at least about 20000:1, 10000:1, 7500:1, 5000:1, 2500:1, 1000:1, 500:1, 250:1, 100:1. In some embodiments, the complex is made in water (e.g., water at physiological pH).

In another embodiment, the polyglucosamine derivative/therapeutic agent complex is made (e.g., formed) by premixing a therapeutic agent (e.g., a small molecule, peptide, or protein described herein) with a polyglucosamine derivative (e.g., a polyglucosamine derivative described herein (e.g., polyglucosamine-arginine), e.g., at a weight ratio (polyglucosamine derivative:therapeutic agent) of at least about 20000:1, 10000:1, 7500:1, 5000:1, 2500:1, 1000:1, 500:1, 250:1, 100:1, in a medium (e.g., a serum-free medium).

In some embodiments, the therapeutic agent (e.g., a small molecule, peptide, or protein described herein) is added to the medium before the polyglucosamine derivative is added.

In some embodiments, the polyglucosamine derivative is added to the medium before the therapeutic agent (e.g., a small molecule, peptide, or protein described herein) is added.

In yet another embodiment, the polyglucosamine derivative/therapeutic agent complex is made (e.g., formed) by sequentially adding a therapeutic agent (e.g., a small molecule, peptide, or protein described herein) and a polyglucosamine derivative (e.g., a polyglucosamine derivative described herein (e.g., polyglucosamine-arginine), e.g., at a weight ratio (polyglucosamine derivative:therapeutic agent) of at least about 20000:1, 10000:1, 7500:1, 5000:1, 2500:1, 1000:1, 500:1, 250:1, 100:1 to the cells. In some embodiments, the therapeutic agent (e.g., a small molecule, peptide, or protein described herein) is added to the cells before the polyglucosamine derivative is added. In some embodiments, the polyglucosamine derivative is added to the cells after the therapeutic agent (e.g., a small molecule, peptide, or protein described herein) is added. In some embodiments, the cells are suspension cultured cells. In one embodiment, the method further comprising the step of adding a lipid or lipid formulation (e.g., a lipid formulation described herein) to the polyglucosamine derivative/therapeutic agent mixture or adding a lipid or lipid formulation (e.g., a lipid formulation described herein) to the cells.

In some embodiments, the derivatized polyglucosamines and therapeutic agents, e.g., small molecules, peptides, or proteins, form a non-covalently-bonded complex, e.g., they associate non-covalently, e.g., electrostatic attraction between the derivatized polyglucosamines and the therapeutic agents stabilize the complex. For example, in a complex of poly(acetyl, arginyl) glucosamine (PAAG) and phenytoin, the positively charged PAAG and the negatively charged phenytoin associate non-covalently due to electrostatic attraction between the species.

### Nanoparticle Complexes

Methods, compounds and compositions described herein are useful for the formation and use of a nanoparticle complex of controllable size having a composition including the polyglucosamine derivative and therapeutic agent (e.g., a small molecule, peptide, or protein described herein). The nanoparticle complexes may include but are not limited to coprecipitate(s) or coacervate such as sodium sulfate or tripolyphosphate (TPP) salt. The nanoparticle complexes are taken up by a cell where the therapeutic agent (e.g., a small molecule, peptide, or protein described herein) is therein released in a desirable timeframe.

### Compositions and Complexes

Methods, complexes and compositions for binding and delivering a small molecule, peptide, or protein, e.g., to a cell (e.g., a cancer cell), an enzyme, or tissue (e.g., mucosal membrane and epithelial membrane) are described herein. Small molecules, peptides, and proteins may be delivered in vivo or in vitro. Accordingly, compositions and complexes for small molecules, peptides, or proteins are described herein.

In some embodiments, a composition for delivering a small molecule, peptide, or protein includes a functionalized polyglucosamine-arginine described herein, e.g., a compound of formula (I). The positively charged moieties on the polymer serve to effectively bind the negatively charged small molecule, peptide, or protein. In some embodiments, a complex for delivering a small molecule, peptide, or protein is formed which comprises a functionalized polyglucosamine-arginine described herein, e.g., a compound of formula (I), and a small molecule, peptide, or protein, e.g., a small molecule, peptide, or protein described herein. The positively charged moieties on the polymer serve to effectively bind the negatively charged small molecule, peptide, or protein.

In some embodiments, the composition includes a small molecule, peptide, or protein, e.g., a small molecule, peptide, or protein described herein. In some embodiments, the composition comprises a complex including a small molecule, peptide, or protein, e.g., a small molecule, peptide, or protein described herein.

In some embodiments, the composition is a pharmaceutical composition.

In some embodiments, the composition includes a compound that is used to promote delivery of small molecules, peptides, or proteins. Such compounds may include a peptide or protein transfection reagent described herein.

In some embodiments, the composition includes a precipitating solution, which may include salts such as sodium sulfate or a tripolyphosphate (TPP) salt. The pH, ionic strength and temperature of the precipitating solutions can be adjusted for optimization of binding and delivery, the range of DNA incorporation at pH 7 with minimal coprecipitating factors is facilitated and optimized by incorporation of the described positively charged polyglucosamine derivatives. Due to the solubility of the polyglucosamine derivatives at a range of molecular weights and degrees of functionalization, optimization of a delivery strategy for a variety of nucleic acid types and sizes is facilitated.

The complexes and compositions described herein can be formulated in a variety of manners, including for topical, enteral, or parenteral delivery. For example, the complexes can be administered, e.g., topically (e.g., by solution (e.g., oral rinse, throat gargle, eye drop), lotion, cream, ointment, gel, foam, transdermal patch, powder, solid, ponge, tape, vapor, inhalation, or intranasal spray (e.g., nasal spray, nasal mists, sinus spray, nebulizer), enema, eye drops), enterally (e.g., orally, gastric feeding tube, duodenal feeding tube, gastrostomy, rectally, buccally), or pareterally (e.g., intravenously, intra-arterially). In some embodiments, inhalation sprays (e.g., nasal spray, nasal mists, or sinus spray), are used for the nasal delivery of a complex or composition described herein, to locally treat a disorder or condition described herein. Inclusion in feed, water or an inhaled formulation is particularly desirable for use with animals. In some embodiments, a complex or composition is formulated so as to allow the soluble polyglucosamine derivative thereof to diffuse into a subject (e.g., into the wound, body cavities, mucosal membrane, or epithelia membrane of a subject) upon administration to the subject or to be ingested, inhaled or swabbed while incorporated into a time release formulation.

In an embodiment, the compositions described herein can be formulated, e.g., as a solution, encapsulated time release, gel, ointment, dressing, spray, powder, or lavage, to deliver a therapeutic agent described herein (e.g., a small molecule, peptide, or protein) to a cell (e.g., cancer cell) or tissue (e.g., mucosal membrane or epithelia membrane), e.g., to treat a disorder or condition or a symptom thereof (e.g., pain, inflammatory disorder, proliferative disorder (e.g., cancer), dermal disorder or condition (e.g., wound)). In an embodiment, the dosage (e.g., solution) is from about 10 μg/mL to about 1000 μg/mL, about 100 μg/mL to about 750 μg/mL, or about 250 μg/mL to about 500 μg/mL. In an embodiment, the dosage (e.g., dressing) is from about 1% to about 10%, about 3% to about 8%, or about 5% to about 6%, by weight. In an embodiment, the composition is applied to a thickness of at least about 1/128, 1/64, 1/32, or 1/16 inch. In an embodiment, the dosage (e.g., solid diffusible preparation) is from about 1% to about 20%, about 2% to about 15%, or about 5% to about 10%, by weight. In an embodiment, the dosage (e.g., solid dissolvable preparation) is from about 2% to about 95%, about 5% to about 90%, about 10% to about 80%, about 20% to about 70%, about 30% to about 60%, or about 40% to about 50%, by weight. In an embodiment, the dosage is from about 1 mg/kg to about 100 mg/kg, about 2 mg/kg to about 75 mg/kg, about 5 mg/kg to about 50 mg/kg, or about 10 mg/kg to about 25 mg/kg body weight, e.g., in an encapsulated time release, gel, capsule, or enema. In an embodiment, the composition is administered at least 1, 2, 3, 4, 5 or 6 times daily.

Lower or higher doses than those recited above may be required. Specific dosage and treatment regimens for any particular patient will depend upon a variety of factors, including the activity of the specific complex or composition employed, the age, body weight, general health status, sex, diet, time of administration, rate of excretion, drug combination, the severity and course of the disease, condition or symptoms, the type and nature of the bacteria, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

Pharmaceutical compositions of this invention comprise a complex or composition of the formulae described herein or a pharmaceutically acceptable salt thereof; an additional compound including for example, a steroid or an analgesic; and any pharmaceutically acceptable carrier, adjuvant or vehicle. Alternate compositions of this invention comprise a complex described herein or a pharmaceutically acceptable salt thereof; and a pharmaceutically acceptable carrier, adjuvant or vehicle. The compositions delineated herein include the complexes described herein, as well as additional therapeutic complexes if present, in amounts effective for achieving a modulation of disease or disease symptoms.

The compositions are generally made by methods including the steps of combining a complex or composition described herein with one or more carriers and, optionally, one or more additional therapeutic compounds delineated herein.

The term “pharmaceutically acceptable carrier or adjuvant” refers to a carrier or adjuvant that may be administered to a patient, together with a complex or composition of this invention, and which does not destroy the pharmacological activity thereof and is nontoxic when administered in doses sufficient to deliver a therapeutic amount of the compound.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase which can be combined with emulsifying and/or suspending agents. If desired, certain sweetening and/or flavoring and/or coloring agents may be added.

The complexes and compositions of this invention may be administered by aerosol, nebulizer, or inhalation. In some embodiments, the composition is in the form of a dry powder, a suspension, or a solution. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Exemplary methods and devices for aerosol or inhalation include those described in U.S. Pat. No. 6,962,151, which is incorporated herein by reference in its entirety.

Compositions formulated for inhaled delivery generally include particles having a mean diameter of from about 0.1 μm to about 50 μm (e.g., from about 0.1 μm to about 10 μm, or from about 0.2 μm to about 5 μm. In some embodiments, the composition includes a dispersion of suitably-sized dry particles, for example, precipitants or crystals) or a dispersion of a solution (e.g., droplets) of a suitable size.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some cases, the pH of the formulation may be adjusted with pharmaceutically acceptable acids, bases or buffers to enhance the stability of the formulated composition or its delivery form for delivery in particular regions of the body, such as the colon.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solublizing or dispersing agents known in the art.

When the compositions of this invention comprise a combination of compounds described herein, both the compounds are generally present at dosage levels of between about 1 to 100%, and more preferably between about 5 to 95% of the dosage normally administered in a monotherapy regimen. Additionally, combinations of a plurality of compounds described herein are also envisioned. The compounds may be administered separately, as part of a multiple dose regimen, from the compounds of this invention. The compounds may be administered in a manner and dose where they act synergistically. Alternatively, those compounds may be part of a single dosage form, mixed together with the compounds of this invention in a single composition.

### Kits

A complex or composition described herein can be provided in a kit. The kit includes (a) a complex or composition that includes a compound described herein, and, optionally (b) informational material. The informational material can be descriptive, instructional, marketing or other material that relates to the methods described herein and/or the use of the complex or composition described herein for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to use of the complex or composition described herein to treat a disorder described herein.

In one embodiment, the informational material can include instructions to administer the complex or composition described herein in a suitable manner to perform the methods described herein, e.g., in a suitable dose, dosage form, or mode of administration (e.g., a dose, dosage form, or mode of administration described herein). In some embodiments, the doses, dosage forms, or mode of administration can be, e.g., transdermal or transmucosal. Preferred doses, dosage forms, or modes of administration are e.g., topical (e.g., epicutaneous, intradermal, subcutaneous, sublingual, bucosal, inhalational, eye drops, ear drops), enteral (e.g., oral, gastrointestinal, rectal), parenteral (e.g., intravenous, intra-arterial, intramuscular). In another embodiment, the informational material can include instructions to administer the complex or composition described herein to a suitable subject, e.g., a human, e.g., a human having or at risk for a disorder described herein. For example, the material can include instructions to administer the complex or composition described herein to such a subject.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as computer readable material, video recording, or audio recording. In another embodiment, the informational material of the kit is contact information, e.g., a physical address, email address, website, or telephone number, where a user of the kit can obtain substantive information about a complex or composition described herein and/or its use in the methods described herein. The informational material can also be provided in any combination of formats.

In addition to a complex or composition described herein, the composition of the kit can include other ingredients, such as a solvent or buffer, a stabilizer, a preservative, and/or a second complex or composition for treating a condition or disorder described herein. Alternatively, the other ingredients can be included in the kit, but in different compositions or containers than the complex or composition described herein. In such embodiments, the kit can include instructions for admixing the complex or composition described herein and the other ingredients, or for using a complex or composition described herein together with the other ingredients.

The complex or composition described herein can be provided in any form, e.g., liquid, dried or lyophilized form. It is preferred that the complex or composition described herein be substantially pure and/or sterile. When the complex or composition described herein is provided in a liquid solution, the liquid solution preferably is an aqueous solution, with a sterile aqueous solution being preferred. When the complex or composition described herein is provided as a dried form, reconstitution generally is by the addition of a suitable solvent. The solvent, e.g., sterile water or buffer, can optionally be provided in the kit.

The kit can include one or more containers for the composition containing the complex or composition described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a complex or composition described herein. For example, the kit includes a plurality of syringes, ampules, foil packets, or blister packs, each containing a single unit dose of a complex or composition described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is an implantable delivery device.

### Treatment

The compositions and compounds described herein can be administered to a tissue, e.g. in vitro or ex vivo, or to a subject, e.g., in vivo, to treat and/or prevent a variety of disorders or conditions, or symptoms thereof, including those described herein below.

As used herein, the term “treat” or “treatment” is defined as the application or administration of a composition or complex (e.g., a composition or complex described herein) to a subject, e.g., a patient, or application or administration of the composition or complex or composition to an isolated tissue, from a subject, e.g., a patient, who has a disorder or condition (e.g., a disorder or condition described herein), a symptom of a disorder or condition, or a predisposition toward a disorder or condition, with the purpose to cure, heal, alleviate, relieve, alter, remedy, ameliorate, improve or affect the disorder or condition, one or more symptoms of the disorder or condition, or the predisposition toward the disorder or condition (e.g., to prevent at least one symptom of the disorder or condition or to delay onset of at least one symptom of the disorder or condition), and/or a side or adverse effect of a therapy, e.g., a cancer therapy.

As used herein, the term “prevent” or “prevention” is defined as the application or administration of a complex or composition (e.g., a complex or composition described herein) to a subject, e.g., a subject who is at risk for a disorder or condition (e.g., a disorder or condition described herein), or has a disposition toward a disorder or condition, or application or administration of the complex or composition to an isolated tissue from a subject, e.g., a subject who is at risk for a disorder or condition (e.g., a disorder or condition described herein), or has a predisposition toward a disorder or condition, with the purpose to avoid or preclude the disorder or condition, or affect the predisposition toward the disorder or condition (e.g., to prevent at least one symptom of the disorder or condition, or to delay onset of at least one symptom of the disorder or condition).

As used herein, an “amount of a composition or complex effective to treat a disorder or condition” or a “therapeutically effective amount” refers to an amount of the composition or complex which is effective, upon single or multiple dose administration to a subject, in treating a tissue, or in curing, alleviating, relieving or improving a subject with a disorder or condition beyond that expected in the absence of such treatment.

As used herein, an “amount of a composition or complex effective to prevent a disorder” or “a prophylactically effective amount” of the composition or complex refers to an amount effective, upon single- or multiple-dose administration to the subject, in preventing or delaying the occurrence of the onset or recurrence of a disorder or condition, or a symptom of the disorder or condition.

The compounds or compositions described herein can be administered before, during or after the onset of the disorder or condition described herein. For example, the compounds or compositions described herein can be administered in a subject who has been treated or is being treated for a disorder or condition or a symptom thereof described herein, with one or more therapies, e.g., analgesic, anti-inflammatory therapy, anti-cancer therapy (e.g., chemotherapy or radiation therapy), dermal therapy, and wound therapy. The methods herein contemplate administration of an effective amount of the complex or composition to achieve the desired or stated effect. Typically, the complex or composition of this invention will be administered from about 1 to 12 times, about 1 to 10 times, about 1 to 8 times, about 1 to 6 times, about 1 to 4 times, about 1 to 2 times, about 3 to 12 times, about 5 to 12 times, about 7 to 12 times, about 9 to 12 times per day. Alternatively, the complex or composition can be administered as a continuous time-release. Such administration can be used as a chronic or acute therapy. The amount of active ingredient that may be combined with the carrier materials to produce a single dosage form will vary depending upon the host treated and the particular mode of administration.

As used herein, “administered in combination” or a combined administration of two agents means that two or more agents (e.g., compounds or compositions described herein) are administered to a subject at the same time or within an interval such that there is overlap of an effect of each agent on the patient. Preferably they are administered within 15, 10, 5, or 1 minute of one another. Preferably the administrations of the agents are spaced sufficiently close together such that a combinatorial (e.g., a synergistic) effect is achieved. The combinations can have synergistic effect when used to treat a subject having a bacterial infection. The agents can be administered simultaneously, for example in a combined unit dose (providing simultaneous delivery of both agents). Alternatively, the agents can be administered at a specified time interval, for example, an interval of minutes, hours, days or weeks. Generally, the agents are concurrently bioavailable, e.g., detectable, in the subject.

In a preferred embodiment, the agents are administered essentially simultaneously, for example two unit dosages administered at the same time, or a combined unit dosage of the two agents. In another preferred embodiment, the agents are delivered in separate unit dosages. The agents can be administered in any order, or as one or more preparations that includes two or more agents. In a preferred embodiment, at least one administration of one of the agents, e.g., the first agent, is made within minutes, one, two, three, or four hours, or even within one or two days of the other agent, e.g., the second agent. In some cases, combinations can achieve synergistic results, e.g., greater than additive results, e.g., at least 1.25, 1.5, 2, 4, 10, 20, 40, or 100 times greater than additive.

### Subject

The subject can be a human or a non-human animal. Suitable human subjects includes, e.g., a human patient having a disorder or condition, or a symptom of a disorder or condition, e.g., a disorder or condition described herein (e.g., pain, inflammatory disorder, proliferative disorder (e.g., cancer), dermal disorder or condition (e.g., wound)); or a normal subject. The term “non-human animals” of the invention includes all vertebrates, e.g., non-mammals (such as chickens, amphibians, reptiles) and mammals, such as non-human primates, e.g., elephant, sheep, dog, cat, cow, pig, etc. Suitable animal subjects include: but are not limited to, wild animals, farm animals, zoo animals, circus animals, companion (pet) animals, domesticated and/or agriculturally useful animals. Suitable animal subjects include primates, rodents, and birds. Examples of said animals include, but not limited to, elephants, guinea pigs, hamsters, gerbils, rat, mice, rabbits, dogs, cats, horses, pigs, sheep, cows, goats, deer, rhesus monkeys, monkeys, tamarinds, apes, baboons, gorillas, chimpanzees, orangutans, gibbons, fowl, e.g., pheasant, quail (or other gamebirds), a waterfowl, ostriches, chickens, turkeys, ducks, and geese or free flying bird.

### Delivery to Cells

Methods, compounds and compositions for binding and delivering a small molecule, peptide, or protein described herein, e.g., to a cell (e.g., to the cell surface or within the cell, or to a organelle within a cell) (e.g., cancer cell), are described herein. In some embodiments, a composition, complex, or particle described herein may be delivered in vitro or in vivo via a transfection-like mechanism. As used herein, the term “transfection” or “transfection-like” refers to a process of introducing peptides or proteins into cells, e.g., animal cells, by non-viral methods. Transfection of animal cells typically involves opening transient pores or “holes” in the cell plasma membrane, to allow the uptake of material.

There are various methods of introducing foreign peptides or proteins into a eukaryotic cell. Many materials can be used as carriers for delivery of small molecules, peptides, or proteins within a cell. Exemplary methods of delivery include, for example:

Electroporation: Cells suspended in a buffered solution of the small molecule, peptide or protein of interest are placed in a pulsed electrical field. Brief, high-voltage electric pulses result in the formation of small (nanometer-sized) pores in the cell membrane. Small molecules, peptides or proteins enter the cell via these small pores or during the process of membrane reorganization as the pores close and the cell returns to its normal state.

Microinjection: Microinjection has the advantage of introducing small molecules, peptides, or proteins directly into the cell, thereby bypassing exposure to potentially undesirable cellular compartments such as low-pH endosomes.

Viral protein fusions: Several proteins and small peptides have the ability to transduce or travel through biological membranes independent of classical receptor- or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. The small protein transduction domains (PTDs) from these proteins can be fused to other peptides or proteins to successfully transport them into a cell.

Cationic lipids: Certain lipids such as cationic lipids, when placed in an aqueous solution and sonicated, form closed vesicles consisting of a circularized lipid bilayer surrounding an aqueous compartment. These vesicles or liposomes can be formed in a solution containing the peptides or proteins to be delivered.

Exemplary transfection reagents/kits for small molecules, peptides and/or proteins include, but not limited to, Pro-Ject™ Protein Transfection Reagent (Thermo Scientific), TransPass™ P Protein Transfection Reagent (New England Biolabs), Lipodin-Pro™ (Abbiotec), Chariot™ Protein Delivery Reagent (Active Motif), ProteoJuice™ Protein Transfection Reagent (EMD), and TurboFect™ Protein Transfection Reagent (Fermentas).

In some embodiments, the composition described herein further comprises a transfection reagent described herein. For example, the polyglucosamine derivative described herein can increase the efficiency of delivery of a small molecule, peptide, or protein to or within a cell with the transfection reagent by at least 0.5, 1, 2, 5, 10, 20, 50, 100, 500, or 1000 fold, compared to the delivery efficiency of the cell with the transfection reagent in the absence of the derivatized polyglucosamine described herein.

### Activation

Methods, compounds and compositions for binding and delivering a small molecule, peptide, or protein described herein, e.g., to a cell are described herein. In some embodiments, the delivery of a small molecule, peptide, or protein described herein to a cell activates a biological pathway, thereby resulting in the desired effect, e.g., alleviation of symptoms, reduction in pain, inhibition of cell growth, inhibition of vascular formation, etc. In some embodiments, delivery of a small molecule, peptide, or protein to a receptor on the surface of a cell activates a pathway within the cell causing the cell of modify its behavior. In other embodiments, delivery of a small molecule, peptide, or protein to a receptor on the surface of a cell activates a pathway which allows the cell to bind to other receptors or otherwise change the character of the surface of the cell.

In alternative embodiments, the small molecule, peptide, or protein may be delivered to an organelle or a protein, e.g., an enzyme, e.g., a kinase, thereby modifying the protein so that it activates a function, e.g., increasing phosphorylation, or inactivates a function, e.g., decreasing phosphorylation.

### EXAMPLES

EGF receptors are integral membrane proteins with an extracellular ligand binding domain and intracellular tyrosine kinase. Upon binding ligand the receptors dimerize thus bringing the kinase domains together leading to phosphorylation of tyrosine residues within the receptor intracellular domain. These phosphorylated tyrosine residues act as docking sites for a number of proteins and binding to the activated receptor results in the activation of several downstream signaling pathways. One well characterized pathway involves the phosphorylation and activation of the extracellular regulated kinases 1 and 2 (ERK 1/2), also known as mitogen activated protein kinases (p42/p44 MAPK). The level of ERK phosphorylation correlates with its level of activation; therefore we measure ERK phosphorylation as a surrogate marker for EGF receptor and downstream pathway activation. Examples 1-3 illustrate the effect of polyglucosamine derivatives on EGF-induced signaling.

### Example 1

### Poly (Acetyl, Arginyl) Glucosamine (PAAG) Enhances the Ability of Submaximal EGF Concentration to Induce Activation of ERK in Caco2 Cells

Caco2 intestinal epithelial cells were cultured in 96-well tissue culture plates for 5 days in serum containing medium until cells were mostly confluent. Serum containing medium was replaced with serum free medium for one hour before cells were stimulated. 20 μl of 2 mg/ml_poly (acetyl, arginyl) glucosamine (PAAG)_(30 kD, 28% functionalized) was added to 20 μl of 0.2 or 2μ/ml EGF and incubated together at room temperature for one hour. Cell were treated by adding poly (acetyl, arginyl) glucosamine to a final concentration of 100 μg/ml, EGF alone at a final concentration of 10 or 100 ng/ml, or the combination of EGF and PAAG)_at the same final concentrations but incubated together before adding to the cells. After 10 minutes of treatment the medium was aspirated and the cells were lysed in 50 μl of lysis buffer from the AlphaScreen® SureFire® Phospho-ERK 1/2 assay kit (Perkin Elmer). The plate was gently agitated for 10 minutes before 4 μl aliquots of each sample were added to duplicate wells of 384 well white proxiplate. The level of ERK phosphorylation was assayed using the SureFire® Phospho-ERK 1/2 assay kit and measured using the AlphaScreen® settings on an Envision plate reader (Perkin Elmer).

AlphaScreen™ SureFire™ (PerkinElmer) is an immuno-sandwich based assay that provides a quantitative method to measure activation of cellular proteins. Briefly, an antibody that recognizes non-activated epitope of the target protein is coupled with a donor bead, and a second antibody that specifically recognizes the active form of the target protein is coupled to an acceptor bead. A signal is emitted when the donor and acceptor are brought into close proximity by binding the same protein. The magnitude of the signal is directly proportional to the amount of activated protein present in the sample. Signals will be measure using the AlphaScreen™ settings (excitation at 680 nm, emission at 520-620 nm) on the Envision plate reader (PerkinElmer).

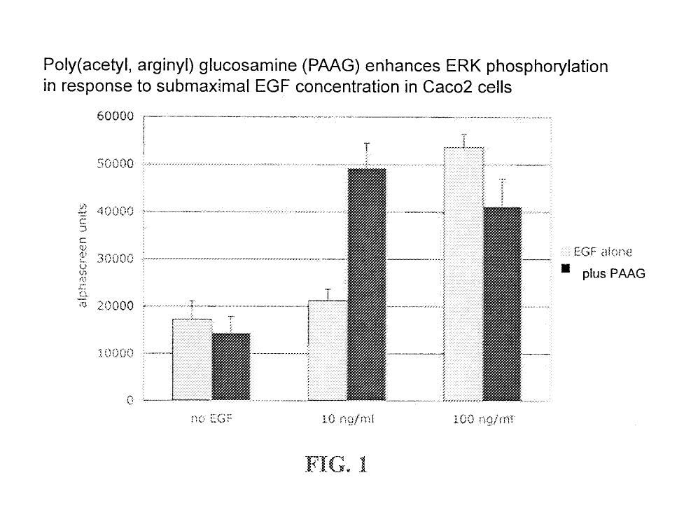

Data shown in FIG. 1 are from one experiment in which each condition was carried out in triplicate wells of the 96 well tissue culture plate and each well was assayed in duplicate. These results indicate that polyglucosamine-arginine enhanced the ability of submaximal EGF concentration to induce activation of ERK in Caco2 cells.

### Example 2

### Effects of Poly (Acetyl, Arginyl) Glucosamine (PAAG) on EGF-Induced Signaling are Mediated Through EGFR and ERK Activation

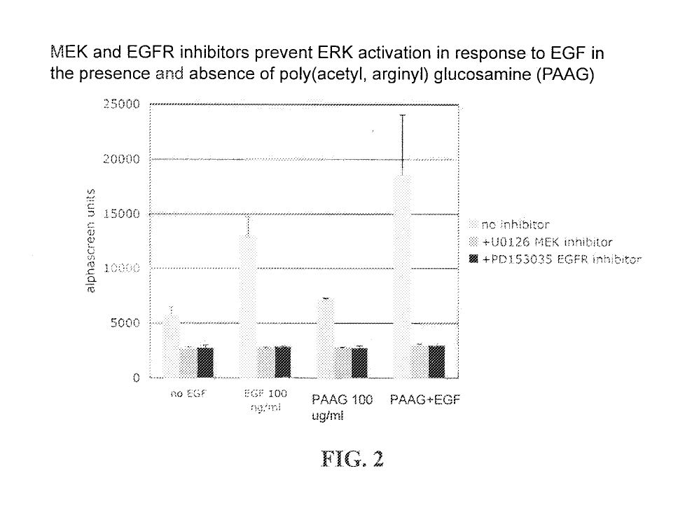

Caco2 intestinal epithelial cells were cultured in 96-well tissue culture plates for 8 days in serum containing medium until cells were mostly confluent and serum containing medium was replaced with serum free medium overnight. Cells were treated with U01260 (10 μM) or PD 153035 (1 μM) for 30 minutes prior to any other treatment. EGF (100 ng/ml), poly (acetyl, arginyl) glucosamine (PAAG)_(30 kD, 28% functionalized) (100 μg/ml) were added to cells alone or in combination with the PAAG added 5 minutes before the EGF. After 5 minutes the medium was aspirated and the cells were lysed in 50 μl of lysis buffer from the AlphaScreen® SureFire® Phospho-ERK 1/2 assay kit (Perkin Elmer). The plate was gently agitated for 10 minutes before 4 μl aliquots of each sample were added to duplicate wells of 384 well white proxiplate. The level of ERK phosphorylation was assayed using the SureFire® Phospho-ERK 1/2 assay kit and measured using the AlphaScreen® settings on an Envision plate reader (Perkin Elmer). Data shown in FIG. 2 are from one experiment in which each condition was carried out in duplicate wells of the 96 well tissue culture plate and each well was assayed in duplicate. These results indicate that the effects of PAAG on EGF-induced signaling are mediated through EGFR and ERK activation.

### Example 3

### Polyglucosamine Derivatives of Different Molecular Weights, Degrees of Functionalization, and Types of Modification Enhance EGF-Induced Signaling

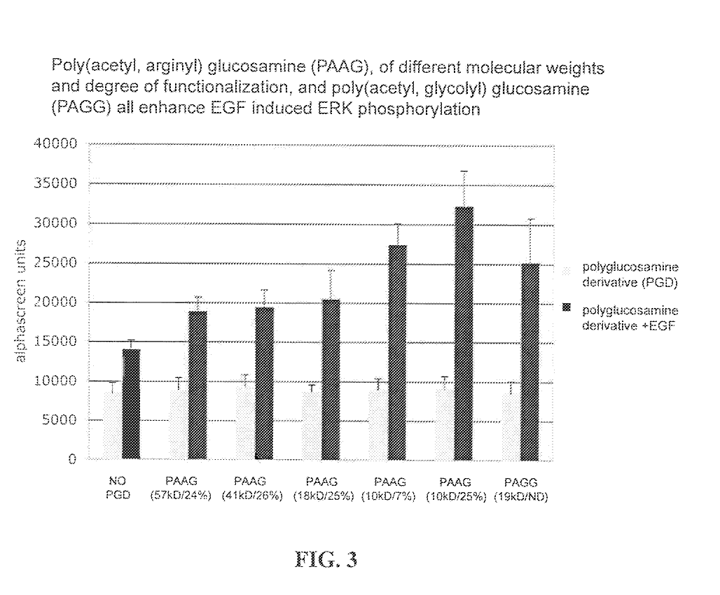

A431 epidermal cells were cultured for one day in 96 well tissue culture plates to achieve confluent monolayers. Serum containing medium was replaced with serum free medium approximately 12 hours before stimulations. Cells were treated with 100 μg/ml of each polyglucosamine derivative (poly (acetyl, arginyl) glucosamine (PAAG) or poly(acetyl, glycolyl) glucosamine (PAGG)) for one hour before addition of 10 ng/ml EGF (submaximal concentration) for 10 minutes. Level of ERK phosphorylation was measured using the AlphaScreen® Surefire® assay (Perkin Elmer). After 10 minutes the medium was aspirated and the cells were lysed in 50 μl of lysis buffer from the AlphaScreen® SureFire® Phospho-ERK 1/2 assay kit (Perkin Elmer). The levels of ERK phosphorylation were assayed using the SureFire® Phospho-ERK 1/2 assay kit as described in the previous examples. Data shown in FIG. 3 are from one experiment in which each condition was carried out in triplicate wells of the 96 well tissue culture plate and each well was assayed in duplicate. These results indicate that polyglucosamine derivatives of different molecular weights, degree of functionalization, and composition, have differing abilities to enhance EGF-induced ERK phosphorylation.

### Example 4

### Addition of Poly (Acetyl, Arginyl) Glucosamine (PAAG) Enhances Cellular Responses to Submaximal TGFβ Concentration

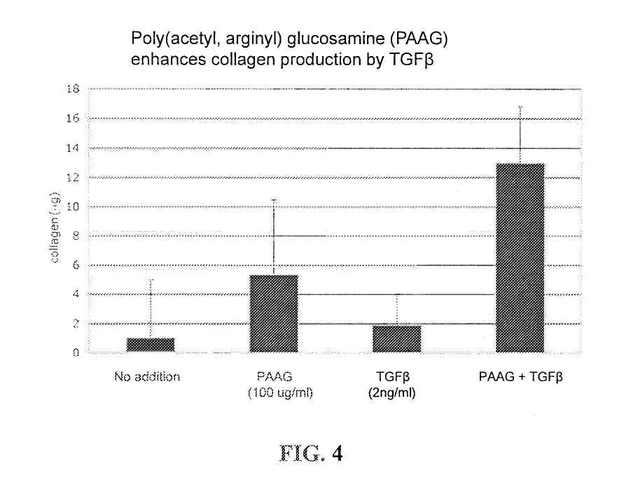

Hs68 human foreskin fibroblasts were seeded into 96 well plates at a density of 4,000 cells per well and cultured in DMEM containing 10% FBS for 24 hours to form confluent monolayers of cells. Serum containing medium was replaced with serum free DMEM and cells were allowed to equilibrate for 2 hours. Cells were then treated by addition of 100 μg/ml poly (acetyl, arginyl) glucosamine (PAAG)(18 kD, 25% functionalization) alone, 2 ng/ml TGFβ alone, or a combination of both with the PAAG being added to the cells immediately prior to the addition of the TGFβ. Cells were incubated with these different treatments for 24 hours before the medium was removed and assayed for the amount of soluble collagen present using the Sircol™ assay (Biocolor). A standard curve was performed using collagen type I. As shown in FIG. 4, addition of PAAG enhanced cellular response to submaximal concentration of TGFβ.

### Example 5

### Arginine Alone has No Effect on Collagen Production in Fibroblasts

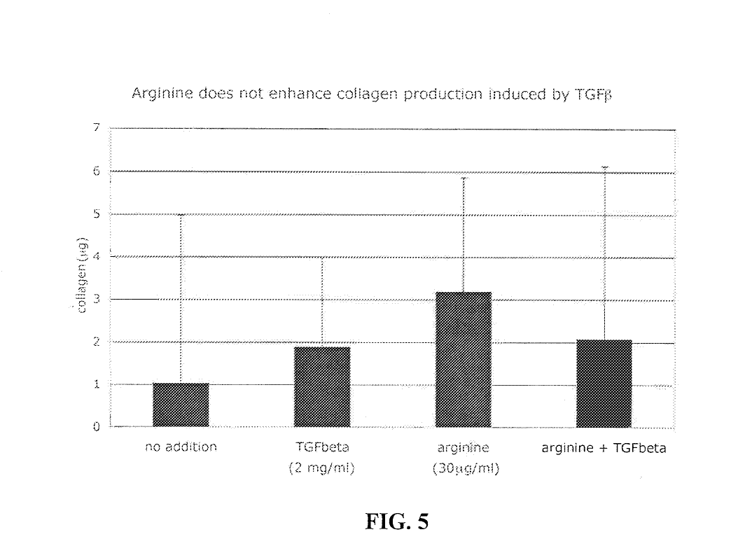

Hs68 human foreskin fibroblasts were seeded into 96 well plates at a density of 4,000 cells per well and cultured in DMEM containing 10% FBS for 24 hours to form confluent monolayers of cells. Serum containing medium was replaced with serum free DMEM and cells were allowed to equilibrate for 2 hours. Cells were treated by addition of 20 μg/ml arginine alone, 2 ng/ml TGFβ alone, or a combination of both with the arginine being added to the cells immediately prior to the addition of TGFβ. Cells were incubated with the different treatment for 24 hours before the medium was removed and assayed for the amount of soluble collagen present using the Sircoff assay (Biocolor). A standard curve was performed using collagen type I. 20 μg/ml arginine is equivalent to the amount of arginine that is present in 100 μg/ml PAAG (18 kD, 25% functionalization). As shown in FIG. 5, addition of arginine alone had no effect on collagen production in fibroblasts.

### Example 6

### Collagen Production in Response to Retinoic Acid is Enhanced with Poly (Acetyl, Arginyl) Glucosamine (PAAG)

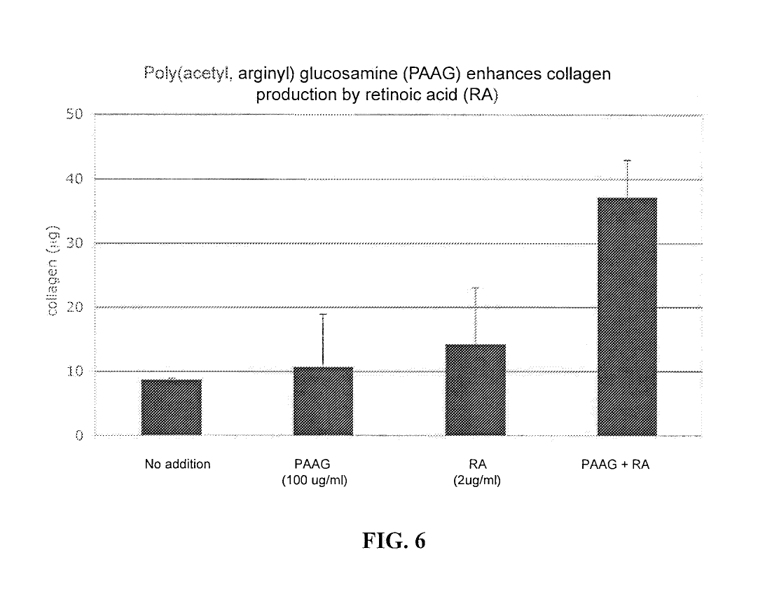

Hs68 human foreskin fibroblasts were seeded into 96 well plates at a density of 4000 cells per well and cultured in DMEM containing 10% FBS for 24 hrs to form confluent monolayers of cells. Serum containing medium was replaced with serum free DMEM and cells allowed to equilibrate for 2 hours. Cells were treated by addition of 100 μg/ml poly (acetyl, arginyl) glucosamine (PAAG)(18 kD, 25% functionalized) alone, 2 μg/ml retinoic acid alone, or a combination of both with the PAAG being added to the cells immediately prior to the addition of the retinoic acid. Cells were incubated with these different treatments for 24 hours before the medium was removed and assayed for the amount of soluble collagen present using the Sircol™ assay (Biocolor). A standard curve was performed using collagen type I. As shown in FIG. 6, PAAG enhanced collagen production in response to retinoic acid.

### Example 7

### Collagen Production in Response to Phenytoin (Dilantin®) is Enhanced with Poly (Acetyl, Arginyl) Glucosamine (PAAG)

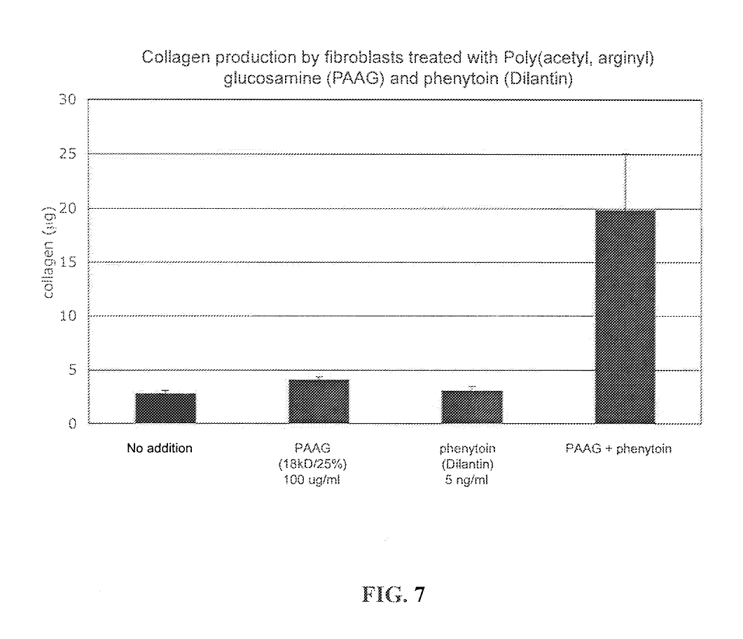

Primary mouse dermal fibroblasts were seeded into 96 well plates at a density of 1,000 cells per well and cultured in DMEM containing 10% FBS for 24 hours to form confluent monolayers of cells. Serum containing medium was replaced with DMEM plus 2% serum (low serum medium) and cells were allowed to equilibrate for 2 hours. Cells were treated by addition of 100 μg/ml poly (acetyl, arginyl) glucosamine (PAAG) (18 kD, 25% functionalized) alone, 5 ng/ml phenytoin alone, or a combination of both with the PAAG being added to the cells immediately prior to the addition of the phenytoin. Cells were incubated with these different treatments for 24 hours before the medium was removed and assayed for the amount of soluble collagen present using the Sircol™ assay (Biocolor). A standard curve was performed using collagen type I. As shown in FIG. 7, PAAG enhanced collagen production in response to phenytoin.

### Example 8

### Polyglucosamine-Arginine is not Toxic to Mammalian Cells in Culture

100 μg/ml poly (acetyl, arginyl) glucosamine (PAAG) (30 kD, 28% functionalization) was added to subconfluent cultures of various mammalian cell lines in 96-well plates. Caco 2 cells are human intestinal epithelial cells, HGF cells are human gingival fibroblasts, Vero cells are green monkey kidney cells, and A431 cells are human epidermal cells. All cells were seeded at least 1 day prior to addition of PAAG and medium was replaced with low serum medium. Cells were incubated for 48 hours and cell numbers were measured using an MTS assay (CellTiter®, Promega) (Caco 2 and HGF cells), CyQUANT® (Invitrogen) (Vero cells) or ATPlite (Perkin Elmer) (A431 cells).

CellTiter 96® Aqueous non-radioactive cell proliferation assay (Promega) is an MTS metabolic assay that measures the conversion of [3-(4,5-dimethylthiazol-2-yl)-5-β-carboxymethoxyphenyl)-2-(4-sulfophenyl)-2H-tetrazolium to a formazan product. The accumulation of the soluble formazan product is monitored by measuring absorbance at 490 nm on an absorbance plate reader (Spectramax plus 384, Molecular Devices).

CyQUANT® cell proliferation assay (Invitrogen) uses a dye that fluoresces strongly when bound to DNA and therefore can be used as a measure of the number of nuclei present. Fluorescent signals will be measured using a fluorescence plate reader (Spectramax Gemini XPS, Molecular Devices) using standard FITC excitation and emission settings (excitation at 485 nm, emission at 530 nm).

ATPlite™ (Perkin Elmer) assay uses luciferase to measure the amount of ATP present in each sample. Emitted light is measured using the luciferase settings on an Envision plate reader.

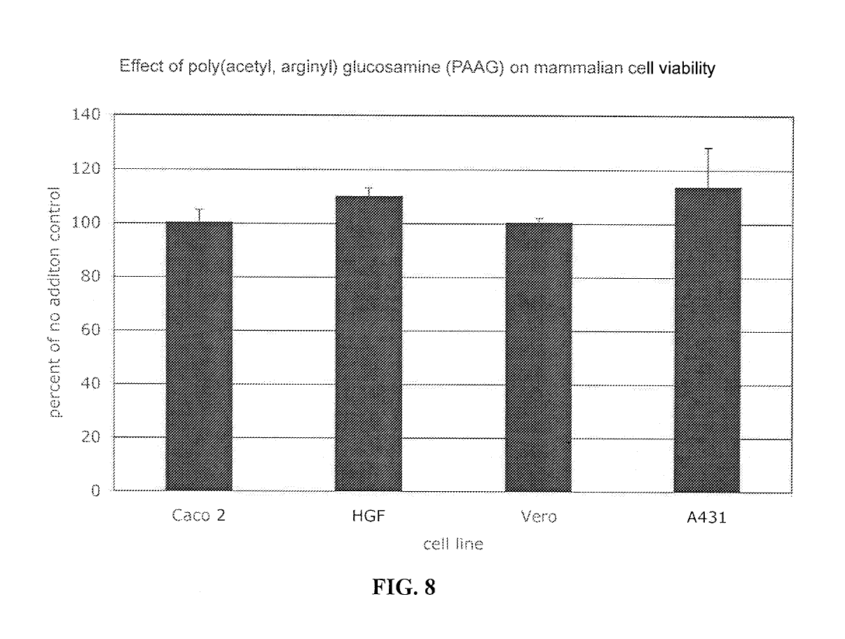

In each of these methods the absolute number of cells can be calculated by creating a standard curve. In the data presented here the signal measured in the treated cells in each assay was compared to the untreated control cells to determine the percent of cells remaining after treatment. As shown in FIG. 8, PAAG was not toxic to cultured Caco2, HGF, Vero, and A431 cells.

1. A method of delivering a small molecule, peptide, or protein to a cell or enzyme, the method comprising:
providing a cell or enzyme;
providing a composition comprising a small molecule, peptide, or protein and a derivatized polyglucosamine of the following formula (I): 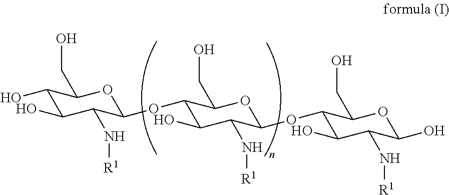
wherein:
n is an integer between 20 and 6000; and
each R¹ is independently selected for each occurrence from hydrogen, acetyl, 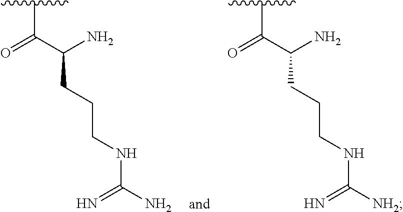 [SMILES: *C(=O)[C@@H](N)CCCNC(=N)N ; "and" ; *C(=O)[C@H](N)CCCNC(=N)N]
wherein at least 25% of R¹ substituents are H, at least 1% of R¹ substituents are acetyl, and at least 2% of R¹ substituents are 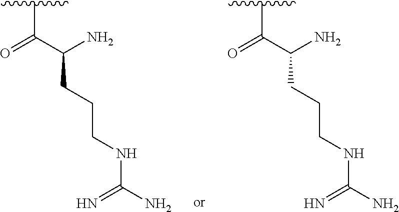 [SMILES: *C(=O)[C@@H](N)CCCNC(=N)N ; "or" ; *C(=O)[C@H](N)CCCNC(=N)N]
, and
contacting the composition with the cell or enzyme,
thereby delivering the small molecule, peptide, or protein to the cell or enzyme.

2. The method of claim 1, wherein the composition is substantially free of polymerized amino acids.

3. (canceled)

4. The method of claim 2, wherein the small molecule, peptide, or protein has a molecular weight of less than about 1000, 750, 500, or 250 Da (e.g., between about 250 Da and about 750 Da).

5. The method of claim 2, wherein the small molecule, peptide, or protein has a molecular weight of greater than about 1, 2, 5, 10, 50, 100, 250, 500, 750, or 1000 kD.

6. The method of claim 2, wherein the small molecule, peptide, or protein comprises an analgesic, an anti-inflammation agent, an anti-epileptic agent, an anti-cancer agent, a cell growth agent, or a wound healing agent.

7. The method of claim 5, wherein the small molecule, peptide, or protein comprises a growth factor or cytokine, e.g., epidermal growth factor (EGF), transforming growth factor beta (TGFβ), interleukin-8 (IL-8).

8-13. (canceled)

14. The method of claim 2, wherein the composition comprises a complex, wherein the complex comprises a small molecule, peptide, or protein and a derivatized polyglucosamine.

15. The method of claim 7, wherein the complex is formed by mixing the small molecule, peptide, or protein and derivatized polyglucosamine in a solution.

16. The method of claim 7, wherein the complex forms a particle, wherein the particle comprises a small molecule, peptide, or protein and a derivatized polyglucosamine.

17. (canceled)

18. The method of claim 2, wherein the derivatized polyglucosamine is soluble in aqueous solution from about pH 3 to about pH 9.

19-28. (canceled)

29. The method of claim 2, wherein the molecular weight of the derivatized polyglucosamine is between 25,000 and 200,000 Da.

30. The method of claim 11, wherein the molecular weight of the functionalized polyglucosamine is between 25,000 and 150,000 Da.

31. The method of claim 12, wherein the molecular weight of the functionalized polyglucosamine is between 25,000 and 110,000 Da.

32. The method of claim 2, wherein the derivatized polyglucosamine is functionalized at between 5% and 50%.

33. The method of claim 14, wherein the derivatized polyglucosamine is functionalized at between 15% and 30%.

34. (canceled)

35. The method of claim 2, wherein the composition achieves a lower therapeutic effective amount than a comparable composition in the absence of the derivatized polyglucosamine, e.g., the lower therapeutic effective amount is at least 10%, 20%, 30%, 40%, 50%, 60%, 70%, 80%, 90%, or 95% less than an amount of the small molecule, peptide, or protein in a comparable composition in the absence of the derivatized polyglucosamine.

36. A method of delivering a small molecule, peptide, or protein to or within a cell or to an enzyme, the method comprising:
providing a cell;
contacting the cell or enzyme with a derivatized polyglucosamine of the following formula (I): 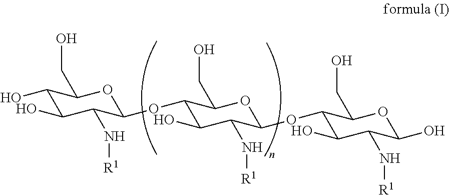
wherein:
n is an integer between 20 and 6000; and
each R¹ is independently selected for each occurrence from hydrogen, acetyl, 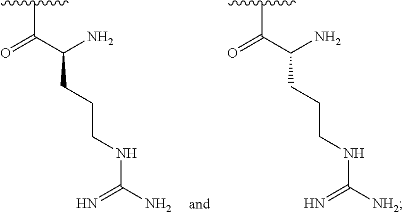 [SMILES: *C(=O)[C@@H](N)CCCNC(=N)N ; "and" ; *C(=O)[C@H](N)CCCNC(=N)N]
wherein at least 25% of R¹ substituents are H, at least 1% of R¹ substituents are acetyl, and at least 2% of R¹ substituents are 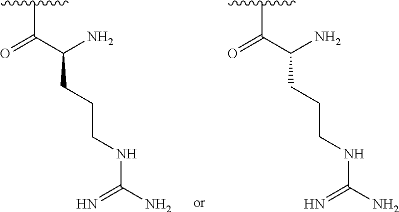 [SMILES: *C(=O)[C@@H](N)CCCNC(=N)N ; "or" ; *C(=O)[C@H](N)CCCNC(=N)N]
, and
contacting the cell or enzyme with the small molecule, peptide, or protein, thereby delivering the small molecule, peptide, or protein to the cell or enzyme.

37. The method of claim 17, wherein the cell or enzyme is contacted with the derivatized polyglucosamine before the cell or enzyme is contacted with the small molecule, peptide, or protein.

38. The method of claim 17, wherein the cell or enzyme is contacted with the small molecule, peptide, or protein before the cell or enzyme is contacted with the derivatized polyglucosamine.

39-48. (canceled)

49. A composition comprising a small molecule, peptide, or protein and a derivatized polyglucosamine of the following formula (I): 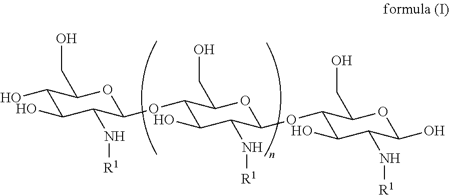
wherein:
n is an integer between 20 and 6000; and
each R¹ is independently selected for each occurrence from hydrogen, acetyl, 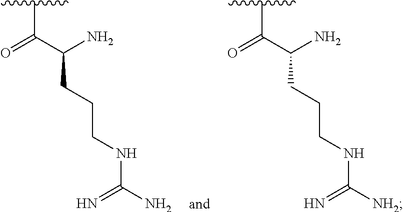 [SMILES: *C(=O)[C@@H](N)CCCNC(=N)N ; "and" ; *C(=O)[C@H](N)CCCNC(=N)N]
wherein at least 25% of R¹ substituents are H, at least 1% of R¹ substituents are acetyl,
and at least 2% of R¹ substituents are

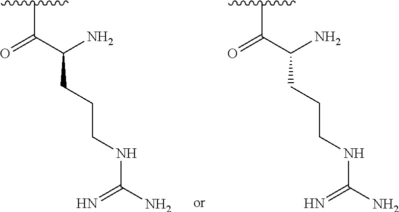 [SMILES: *C(=O)[C@@H](N)CCCNC(=N)N ; "or" ; *C(=O)[C@H](N)CCCNC(=N)N]

50. (canceled)
